(19) United States
(12) Patent Application Publication
(10) Pub. No.: US 2025/0361221 A1
(43) Pub. Date: Nov. 27, 2025

(54) SOLID STATE FORMS OF PALTUSOTINE AND PROCESS FOR PREPARATION THEREOF

(71) Applicant: ASSIA CHEMICAL INDUSTRIES LTD., Tel Aviv (IL)

(72) Inventors: Anantha Rajmohan Muthusamy, Sivakasi (IN); Meenakshi Sundaram Somasundaram, Virudunagar DT (IN); Pramod Kumar Santraj Yadav, Ghaziabad (IN); Vikas Sharma, Greater Noida (IN); Laxman Gande, Suryapet (IN)

(21) Appl. No.: 18/872,457

(22) PCT Filed: Jun. 12, 2023
(86) PCT No.: PCT/IB2023/056041

§ 371 Date: Dec. 6, 2024

(30) Foreign Application Priority Data
Jun. 10, 2022 (IN) 202211033194
Apr. 26, 2023 (IN) 202311030125
May 19, 2023 (IN) 202311035100

(51) Int. Cl. C07D 401/04; A61K 31/4709

(52) U.S. Cl. CPC C07D 401/04; A61K 31/4709

(57) ABSTRACT

The present disclosure encompasses solid state forms of Paltusotine, in embodiments crystalline polymorphs of Paltusotine or salts of Paltusotine, particularly Paltusotine monomesylate and Paltusotine hemimesylate, processes for preparation thereof, and pharmaceutical compositions thereof.

### FIELD OF THE DISCLOSURE

The present disclosure encompasses solid state forms of Paltusotine, in embodiments crystalline polymorphs of Paltusotine or salts of Paltusotine, particularly Paltusotine monomesylate and Paltusotine hemimesylate, processes for preparation thereof, and pharmaceutical compositions thereof.

### BACKGROUND OF THE DISCLOSURE

Paltusotine, 3-(4-(4-aminopiperidin-1-yl)-3-(3,5-difluorophenyl)quinolin-6-yl)-2-hydroxybenzonitrile, has the following chemical structure:

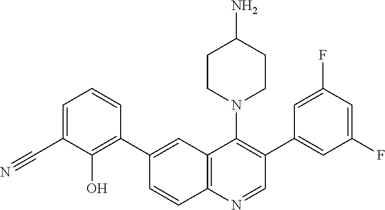 [SMILES: N#Cc1cccc(-c2ccc3ncc(-c4cc(F)cc(F)c4)c(N4CCC(N)CC4)c3c2)c1O]

Paltusotine is an oral non-peptide somatostatin receptor 2 agonist, and it is developed for the treatment of acromegaly, malignant carcinoid syndrome and neuroendocrine tumours.

The compound is described in WO2018/013676. WO2019/143718 discloses crystalline monohydrochloride salt of Paltusotine and WO2021/011641 discloses monomesylate and dimesylate salts of Paltusotine.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Paltusotine.

### SUMMARY OF THE DISCLOSURE

The present disclosure provides crystalline polymorphs of Paltusotine or salts of Paltusotine, particularly Paltusotine monomesylate and Paltusotine hemimesylate, processes for preparation thereof, and pharmaceutical compositions thereof. These crystalline polymorphs can be used to prepare other solid state forms of Paltusotine, Paltusotine salts, particularly Paltusotine monomesylate and Paltusotine hemimesylate and their solid state forms.

The present disclosure also provides uses of the said solid state forms of Paltusotine or salts of Paltusotine, particularly Paltusotine monomesylate and Paltusotine hemimesylate of the present disclosure, in the preparation of other solid state forms of Paltusotine and/or Paltusotine co-crystals and/or salts, particularly Paltusotine monomesylate and Paltusotine hemimesylate thereof, and their solid state forms thereof.

The present disclosure provides crystalline polymorphs of Paltusotine and salts of Paltusotine, particularly Paltusotine monomesylate and Paltusotine hemimesylate for use in medicine, including for the treatment of acromegaly, malignant carcinoid syndrome and neuroendocrine tumours.

The present disclosure also encompasses the use of crystalline polymorphs of Paltusotine and salts of Paltusotine, particularly Paltusotine monomesylate and Paltusotine hemimesylate of the present disclosure for the preparation of pharmaceutical compositions and/or formulations.

In another aspect, the present disclosure provides pharmaceutical compositions comprising crystalline polymorphs of Paltusotine and salts of Paltusotine, particularly Paltusotine monomesylate and Paltusotine hemimesylate according to the present disclosure.

The present disclosure includes processes for preparing the above mentioned pharmaceutical compositions. The processes include combining any one or a combination of the crystalline polymorphs of Paltusotine and salts of Paltusotine, particularly Paltusotine monomesylate and Paltusotine hemimesylate with at least one pharmaceutically acceptable excipient.

The crystalline polymorph of Paltusotine and salts of Paltusotine, particularly Paltusotine monomesylate and Paltusotine hemimesylate as defined herein and the pharmaceutical compositions or formulations of the crystalline polymorph of Paltusotine and salts of Paltusotine, particularly Paltusotine monomesylate and Paltusotine hemimesylate may be used as medicaments, such as for the treatment of acromegaly, malignant carcinoid syndrome and neuroendocrine tumours.

The present disclosure also provides methods of treating acromegaly, malignant carcinoid syndrome and neuroendocrine tumours by administering a therapeutically effective amount of any one or a combination of the crystalline polymorphs of Paltusotine and salts of Paltusotine, particularly Paltusotine monomesylate and Paltusotine hemimesylate of the present disclosure, or at least one of the above pharmaceutical compositions, to a subject suffering from acromegaly, malignant carcinoid syndrome and neuroendocrine tumours, or otherwise in need of the treatment.

The present disclosure also provides uses of crystalline polymorphs of Paltusotine and salts of Paltusotine, particularly Paltusotine monomesylate and Paltusotine hemimesylate of the present disclosure, or at least one of the above pharmaceutical compositions, for the manufacture of medicaments for treating e.g. acromegaly, malignant carcinoid syndrome and neuroendocrine tumours.

### BRIEF DESCRIPTION OF THE DRAWINGS

FIG. 1 shows a characteristic X-ray powder diffraction pattern (XRPD) of Paltusotine Form PL1;

FIG. 2 shows a characteristic X-ray powder diffraction pattern (XRPD) of Paltusotine Form PL11;

FIG. 3 shows a characteristic XRPD of a Paltusotine monomesylate Form PLM1;

FIG. 4 shows a characteristic XRPD of a Paltusotine hemimesylate Form PLM2;

FIG. 5 shows a characteristic XRPD of a Paltusotine Form PL2;

FIG. 6 shows a characteristic XRPD of a Paltusotine Form PL3;

FIG. 7 shows a characteristic XRPD of a Paltusotine Form PL4;

FIG. 8 shows a characteristic XRPD of a Paltusotine Form PL5;

FIG. 9 shows a characteristic XRPD of a Paltusotine Form PL6;

FIG. 10 shows a characteristic XRPD of a Paltusotine Form PL7;

FIG. 11 shows a characteristic XRPD of a Paltusotine Form PL8;

FIG. 12 shows a characteristic XRPD of a Paltusotine Form PL9;

FIG. 13 shows a characteristic XRPD of a Paltusotine Form PL10;

FIG. 14 shows a characteristic XRPD of a Paltusotine Form PL12;

FIG. 15 shows a characteristic XRPD of a Paltusotine Form PL13;

FIG. 16 shows a characteristic XRPD of a Paltusotine Form PL14;

FIG. 17 shows a characteristic XRPD of a Paltusotine Form PL15;

FIG. 18 shows a characteristic XRPD of a Paltusotine Form PL16;

FIG. 19 shows a characteristic XRPD of a Paltusotine Form PL17;

FIG. 20a shows solid state ¹³C NMR spectrum of Form PL1 of Paltusotine (full scan);

FIG. 20b shows solid state ¹³C NMR spectrum of Form PL1 of Paltusotine (at the range of 0-100 ppm);

FIG. 20c shows solid state ¹³C NMR spectrum of Form PL1 of Paltusotine (at the range of 100-200 ppm);

FIG. 21a shows solid state ¹³C NMR spectrum of Form PL11 of Paltusotine (full scan);

FIG. 21b shows solid state ¹³C NMR spectrum of Form PL11 of Paltusotine (at the range of 0-105 ppm); and

FIG. 21c shows solid state ¹³C NMR spectrum of Form PL11 of Paltusotine (at the range of 100-200 ppm).

### DETAILED DESCRIPTION OF THE DISCLOSURE

The present disclosure encompasses solid state forms of Paltusotine, including crystalline polymorphs of Paltusotine, processes for preparation thereof, and pharmaceutical compositions thereof. In embodiments, the present disclosure provides crystalline forms of Paltusotine designated as Form PL1, Form PL2, Form PL3, Form PL4, Form PL4, Form PL6, Form PL7, Form PL8, Form PL9, Form PL10, Form PL11, Form PL12, Form PL13, Form PL14, Form PL15, Form PL16, and Form PL17 (defined herein) and also provides crystalline forms of Paltusotine monomesylate designated as Form PLM1 and Paltusotine hemimesylate designated as Form PLM2 (defined herein).

Solid state properties of Paltusotine and crystalline polymorphs thereof can be influenced by controlling the conditions under which Paltusotine and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Paltusotine described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Paltusotine. In some embodiments of the disclosure, the described crystalline polymorph of Paltusotine may contain from about 1% to about 20% (w/w), from about 5% to about 20% (w/w), or from about 5% to about 10% (w/w) of one or more other crystalline polymorph of the same Paltusotine. For example, a crystalline polymorph of Paltusotine and salts of Paltusotine, particularly Paltusotine monomesylate and Paltusotine hemimesylate according to any aspect or embodiment of the present invention may be polymorphically pure, and may contain: about 20% (w/w) or less, about 10% (w/w) or less, about 5% (w/w) or less, about 2% (w/w) or less, about 1% (w/w) or less, or about 0% of any other forms of Paltusotine and salts of Paltusotine, particularly Paltusotine monomesylate and Paltusotine hemimesylate, respectively, as measured, for example, by XRPD. Thus, for example, a crystalline polymorph of Paltusotine as described in any aspect or embodiment herein, which is polymorphically pure, may contain: about 20% (w/w) or less, about 10% (w/w) or less, about 5% (w/w) or less, about 2% (w/w) or less, about 1% (w/w) or less, or about 0% of any other forms of Paltusotine. As another example, a crystalline polymorph of Paltusotine salt as described in any aspect or embodiment herein, which is polymorphically pure, may contain: about 20% (w/w) or less, about 10% (w/w) or less, about 5% (w/w) or less, about 2% (w/w) or less, about 1% (w/w) or less, or about 0% of any other forms of the Paltusotine salt. Alternatively, a crystalline polymorph of Paltusotine and salts of Paltusotine, particularly Paltusotine monomesylate and Paltusotine hemimesylate according to any aspect or embodiment of the present invention may be polymorphically pure and may contain greater than about 80% (w/w), greater than about 90% (w/w), greater than about 95% (w/w), greater than about 98% (w/w), greater than about 99% (w/w), or about 100% of the crystalline polymorph of Paltusotine and salts of Paltusotine, particularly Paltusotine monomesylate and Paltusotine hemimesylate, respectively.

The solid state forms of Paltusotine (e.g. Paltusotine, Paltusotine salts, or cocrystals) as described in any aspect or embodiment of the present disclosure may be chemically pure, or substantially free of any other compounds.

A compound may be referred to herein as chemically pure or purified compound or as substantially free of any other compounds. As used herein, the terms “chemically pure” or “purified” or “substantially free of any other compounds” refer to a compound that is substantially free of any impurities including enantiomers of the subject compound, diastereomers or other isomers. A chemically pure or purified compound or a compound that is substantially free of any other compound will be understood to mean that it contains about 10% (w/w) or less, about 5% (w/w) or less, about 4% (w/w) or less, about 3% (w/w) or less, about 2% (w/w) or less, about 1.5% (w/w) or less, about 1% (w/w), about 0.8% (w/w) or less, about 0.6% (w/w) or less about 0.4% (w/w) or less about 0.2% (w/w) or less or less, about 0.1% (w/w) or less or about 0% of any other compound as measured, for example, by HPLC. Alternatively, A chemically pure or purified compound or a compound that is substantially free of any other compound will be understood to mean that it contains about 10% area percent or less, about 5% area percent or less, about 4% area percent or less, about 3% area percent or less, about 2% area percent or less, about 1.5% area percent or less, about 1% area percent or less, about 0.8% area percent or less, about 0.6% area percent or less, about 0.4% area percent or less, about 0.2% area percent or less, about 0.1% area percent or less, or about 0% of any other compound as measured by HPLC. Thus, pure or purified Paltusotine and salts of Paltusotine, particularly Paltusotine, Paltusotine monomesylate and Paltusotine hemimesylate described herein as substantially free of any compounds would be understood to contain greater than about 90% (w/w), greater than about 95% (w/w), greater than about 96% (w/w), greater than about 97% (w/w), greater than about 98% (w/w), greater than about 98.5% (w/w), greater than about 99% (w/w), greater than about 99.2%, (w/w) greater than about 99.4% (w/w), greater than about 99.6% (w/w), greater than about 99.8% (w/w), greater than about 99.9% (w/w), or about 100% of the subject Paltusotine. Alternatively, pure or purified Paltusotine and salts of Paltusotine, particularly Paltusotine monomesylate and Paltusotine hemimesylate described herein as substantially free of any compounds would be understood to contain greater than about 90% area percent, greater than about 95% area percent, greater than about 96% area percent, greater than about 97% area percent, greater than about 98% area percent, greater than about 98.5% area percent, greater than about 99% area percent, greater than about 99.2%, area percent, greater than about 99.4% area percent, greater than about 99.6% area percent, greater than about 99.8% (area percent, greater than about 99.9% area percent, or about 100% of the subject Paltusotine and salts of Paltusotine, particularly Paltusotine monomesylate and Paltusotine hemimesylate. The chemical purity may be determined using any suitable process, for example, using HPLC and UV detection (e.g. at 220 nm).

Depending on which other crystalline polymorphs a comparison is made, the crystalline polymorphs of Paltusotine or salts of Paltusotine, particularly Paltusotine monomesylate and Paltusotine hemimesylate of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Paltusotine and salts of Paltusotine, particularly Paltusotine monomesylate and

Paltusotine hemimesylate referred to herein as being characterized by graphical data “as depicted in” or “as substantially depicted in” a Figure will thus be understood to include any crystal forms of Paltusotine characterized with the graphical data having such small variations, as are well known to the skilled person, in comparison with the Figure.

As used herein, and unless stated otherwise, the term “anhydrous” in relation to crystalline forms of Paltusotine, relates to a crystalline form of Paltusotine and salts of Paltusotine, particularly Paltusotine monomesylate and Paltusotine hemimesylate which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

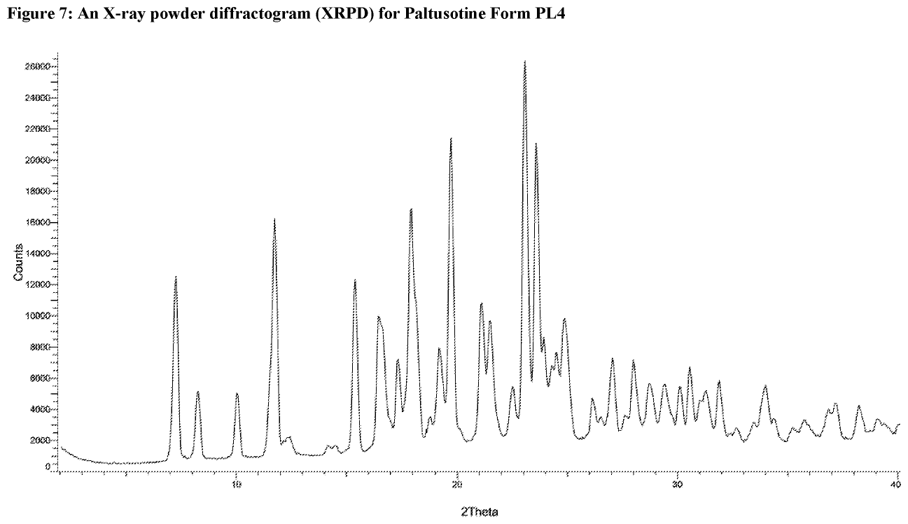

As used herein, Paltusotine Pattern A is described in WO2019/143718, and may be characterized by an XRPD pattern having peaks at 9.2, 12.3, 14.4, and 24.0 degrees 2-theta, or an XRPD pattern having peaks at 9.2, 12.3, 14.4, and 24.0 degrees 2-theta±0.2 degrees 2-theta, preferably wherein the XRPD is measured using Cu Kal radiation having wavelength 1.5406 Å. Alternatively, Paltusotine Pattern A may be characterized by an XRPD having peaks at 9.2, 12.3, 14.4, and 24.0 degrees 2-theta having relative intensities (%) of: 100, 35, 31 and 29, respectively; or an XRPD pattern having peaks at 9.2, 12.3, 14.4, and 24.0 degrees 2-theta±0.2 degrees 2-theta, having relative intensities (%) of: 100, 35, 31 and 29, respectively; preferably wherein the XRPD is measured using Cu Kal radiation having wavelength 1.5406 Å. Alternatively, Paltusotine Pattern A may be described as having a characteristic XRPD pattern substantially as depicted in FIG. 7(a) of WO2019/143718, wherein the XRPD is measured using Cu Kal radiation having wavelength 1.5406 Å.

As used herein, the term “isolated” in reference to crystalline polymorph of Paltusotine and salts of Paltusotine, particularly Paltusotine monomesylate and Paltusotine hemimesylate of the present disclosure corresponds to a crystalline polymorph of Paltusotine and salts of Paltusotine, particularly Paltusotine monomesylate and Paltusotine hemimesylate that is physically separated from the reaction mixture in which it is formed.

As used herein, unless stated otherwise, the XRPD measurements are taken using copper Kα radiation wavelength 1.5418 Å. XRPD peaks reported herein are measured using CuKα radiation, λ=1.5418 Å, typically at a temperature of 25±3° C.

As used herein, unless stated otherwise, solid state ¹³C NMR data is obtained using ¹³C CP/MAS NMR method. Particularly, as used herein, unless stated otherwise, the ¹³C CP/MAS NMR reported herein are measured at 500 MHZ, preferably at a temperature of at 298 K±3° C.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

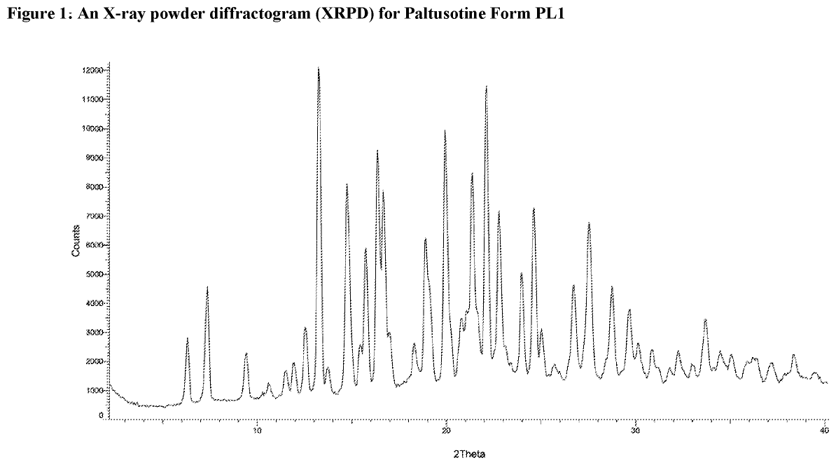

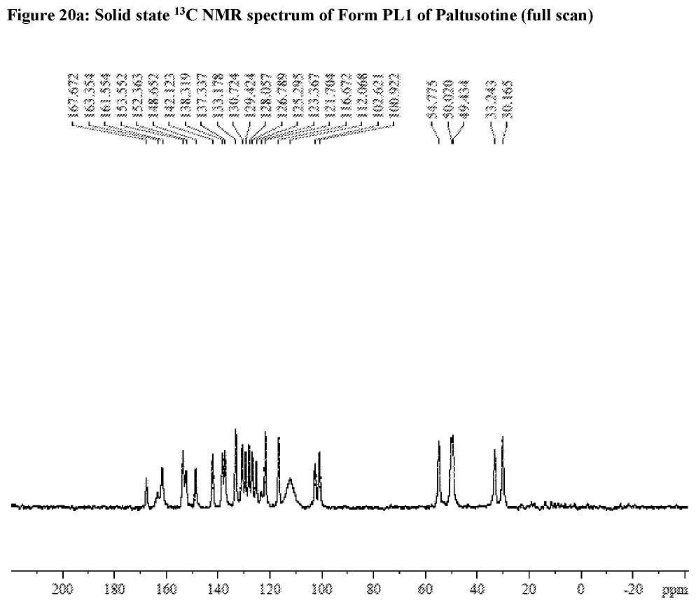

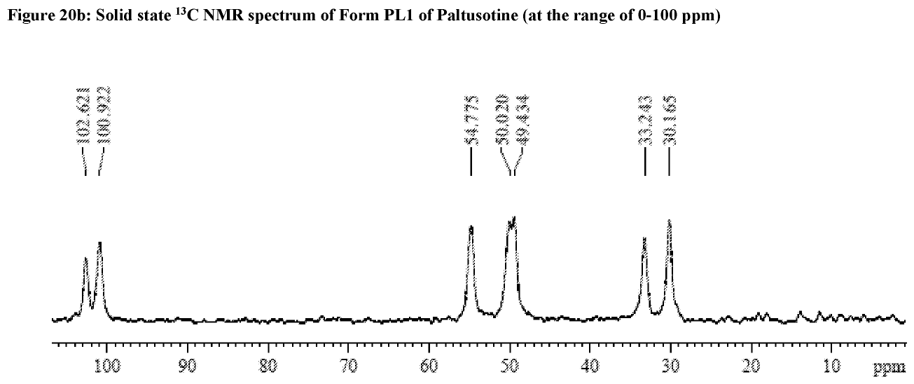

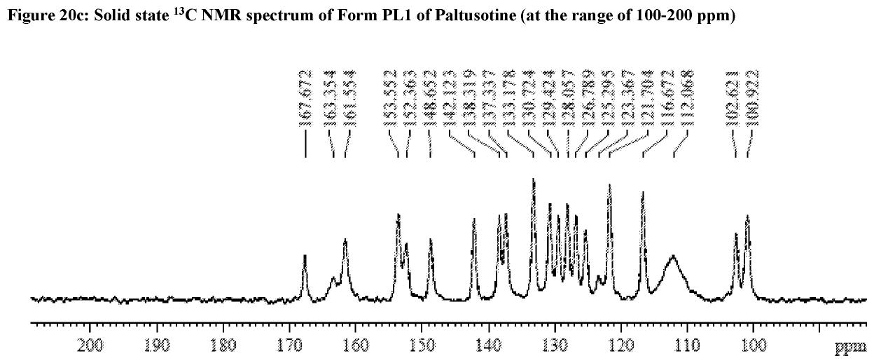

The present disclosure includes a crystalline polymorph of Paltusotine, designated PL1. The crystalline Form PL1 of Paltusotine may be characterized by data selected from one or more of the following: an X-ray powder diffraction pattern substantially as depicted in FIG. 1; an X-ray powder diffraction pattern having peaks at 6.3, 9.5, 14.8, 22.9 and 27.6 degrees 2-theta±0.2 degrees 2-theta; a solid state ¹³C NMR spectrum having peaks at 33.2, 116.7, 129.4, 142.1, 148.7 and 167.7 ppm±0.2 ppm; a solid state ¹³C NMR spectrum having the following chemical shift absolute differences from a reference peak at 102.6 ppm±2 ppm of 69.4, 14.1, 26.8, 39.5, 46.1 and 65.1 ppm±0.1 ppm; a solid state ¹³C NMR spectrum substantially as depicted in FIG. 20a, 20b or 20c; and combinations of these data. Optionally, Form PL1 of Paltusotine according to any aspect or embodiment of the disclosure may be characterized by an X-ray powder diffraction pattern having peaks at 6.3, 9.5, 14.8, 22.9 and 27.6 degrees 2-theta±0.2 degrees 2-theta and an absence of a peak at 8.3 degrees 2-theta±0.2 degrees 2-theta.

Crystalline Form PL1 of Paltusotine may be further characterized by an X-ray powder diffraction pattern having peaks at 6.3, 9.5, 14.8, 22.9 and 27.6 degrees 2-theta±0.2 degrees 2-theta, and also having any one, two, three, four or five additional peaks selected from 11.5, 13.3, 19.9 and 22.1 degrees 2-theta±0.2 degrees 2-theta. Optionally, crystalline Form PL1 of Paltusotine according to any aspect or embodiment of the disclosure may be further characterized by an X-ray powder diffraction pattern having peaks at 6.3, 9.5, 14.8, 22.9 and 27.6 degrees 2-theta±0.2 degrees 2-theta, and also having any one, two, three, four or five additional peaks selected from 11.5, 13.3, 19.9 and 22.1 degrees 2-theta±0.2 degrees 2-theta, and an absence of a peak at 8.3 degrees 2-theta±0.2 degrees 2-theta

Alternatively, crystalline Form PL1 of Paltusotine according to any aspect or embodiment of the disclosure may be characterized by an X-ray powder diffraction pattern having peaks at 6.3, 9.5, 14.8, 22.9 and 27.6 degrees 2-theta±0.2 degrees 2-theta, and also having one additional peak selected from 11.5, 13.3, 19.9 and 22.1 degrees 2-theta±0.2 degrees 2-theta; and optionally an absence of a peak at 8.3 degrees 2-theta±0.2 degrees 2-theta.

Alternatively, crystalline Form PL1 of Paltusotine according to any aspect or embodiment of the disclosure may be further characterized by an X-ray powder diffraction pattern having peaks at 6.3, 9.5, 14.8, 22.9 and 27.6 degrees 2-theta±0.2 degrees 2-theta, and also having two additional peaks selected from 11.5, 13.3, 19.9 and 22.1 degrees 2-theta±0.2 degrees 2-theta; and optionally, an absence of a peak at 8.3 degrees 2-theta±0.2 degrees 2-theta.

Crystalline Form PL1 of Paltusotine may be alternatively characterized by an X-ray powder diffraction pattern having peaks at 6.3, 9.5, 11.5, 13.3, 14.8, 19.9, 22.1, 22.9 and 27.6 degrees 2-theta±0.2 degrees 2-theta; and optionally an absence of a peak at 8.3 degrees 2-theta±0.2 degrees 2-theta.

In one embodiment of the present disclosure, crystalline Form PL1 of Paltusotine is isolated.

Crystalline Form PL1 of Paltusotine may be hydrate form, more preferably hemihydrate. Crystalline Form PL1 of Paltusotine according to any aspect or embodiment of the disclosure may optionally contain from about 1.0 to about 3.0% by weight of water, measured, for example by Karl Fischer analysis, or by TGA.

Crystalline Form PL1 of Paltusotine may be characterized by each of the above characteristics alone/or by all possible combinations, e.g., an XRPD pattern having peaks at 6.3, 9.5, 14.8, 22.9 and 27.6 degrees 2-theta±0.2 degrees 2-theta; an XRPD pattern as depicted in FIG. 1, and combinations thereof.

The present disclosure further comprises a process for preparation of Form PL1 of Paltusotine. This process may comprise:
(i) suspending Paltusotine Pattern A in a solvent;
(ii) stirring the suspension;
(iii) optionally isolating Form PL1 of Paltusotine; and
(iv) optionally drying the Form PL1 of Paltusotine.

In any embodiment of this process, the solvent in step (i) may be propionitrile, cyclohexane, cyclopentyl methyl ether, ethyl acetate and isopropyl acetate (IPAC.) The Paltusotine Pattern A may be suspended at temperature of about 20° C. to about 90° C., about 22° C. to about 80° C., about 24° C. to about 75° C., or about 25° C. to about 60° C. According to any aspect or embodiment of the process, the solvent may be used in an amount of about 10 ml to about 80 ml, about 20 ml to about 70 ml, or about 28 ml to about 70 ml, per gram of Paltusotine Pattern A. Step (ii) comprises stirring the suspension for period of about 15 hour to 60 hours, or about 24 hours to about 48 hours. The PL1 Form of Paltusotine may be isolated, preferably by any suitable process, such as decantation, filtration or by centrifuge, preferably by filtration under vacuum. The filtering may be carried out at a temperature of about 10° C. to about 40° C., about 15° C. to about 30° C., or about 25° C.

In any embodiment of this process, the crystalline Paltusotine Form PL1 may be dried under vacuum. The crystalline Form PL1 of Paltusotine may be dried under vacuum, typically at a temperature of about 40° C. to about 80° C., about 50° C. to about 70° C., about 60° C. The drying may be carried out for any suitable time to remove the solvent, typically about 1 to about 5 hours, about 1.5 hours to about 4 hours, or about 2 hours.

According to any aspect or embodiment of the processes for preparing Paltusotine Form PL1, the process may further comprise combining the Paltusotine Form PL1 with at least one pharmaceutically acceptable excipient to prepare a pharmaceutical composition or dosage form.

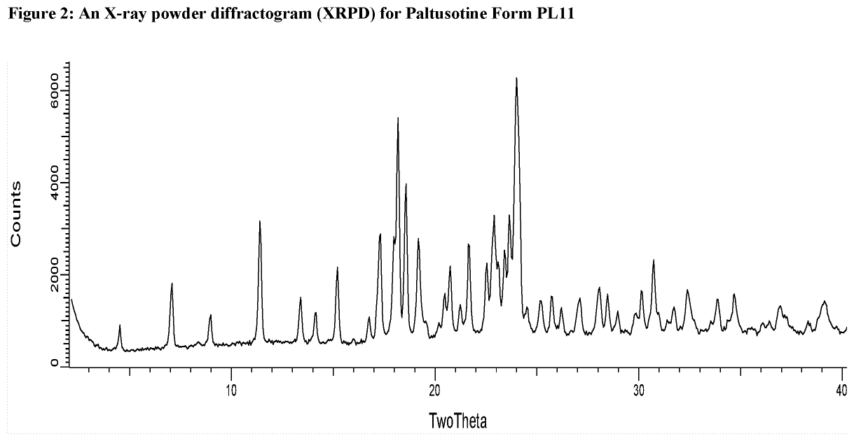

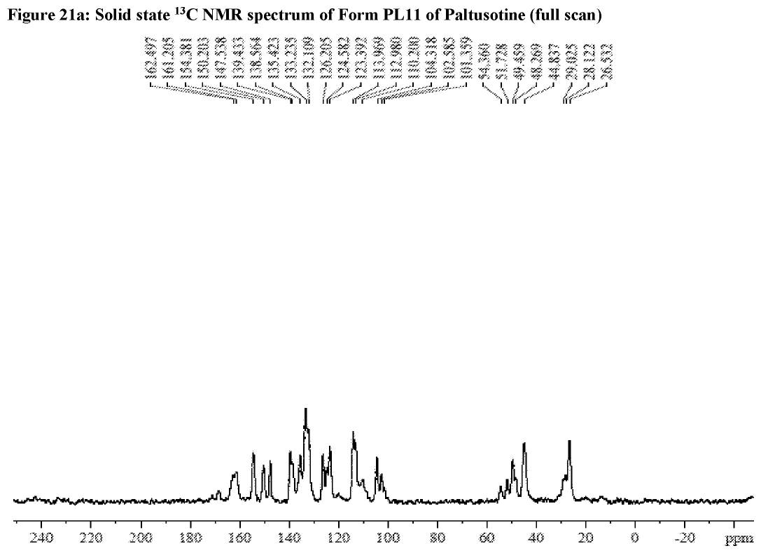

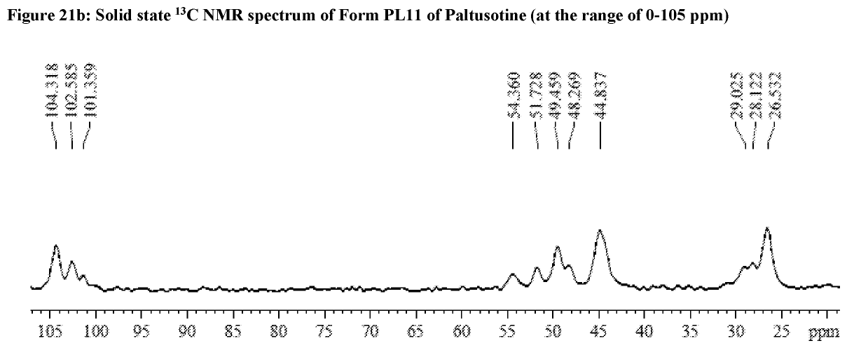

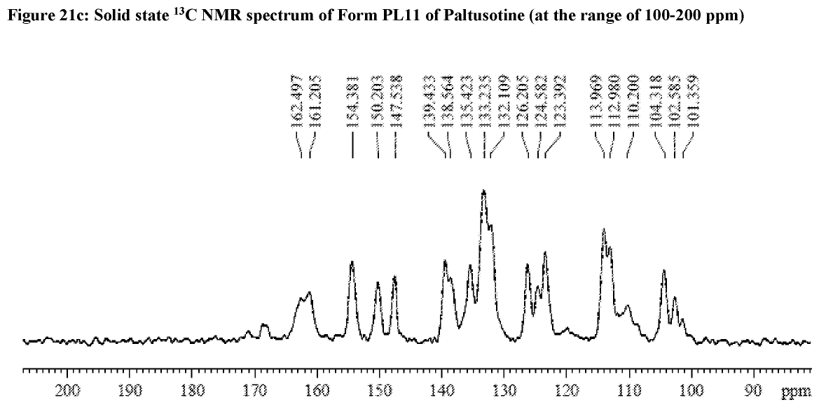

The present disclosure includes a crystalline polymorph of Paltusotine, designated PL11. The crystalline Form PL11 of Paltusotine may be characterized by data selected from one or more of the following: an X-ray powder diffraction pattern substantially as depicted in FIG. 2; an X-ray powder diffraction pattern having peaks at 4.5, 7.1, 15.2, 18.6 and 21.7 degrees 2-theta±0.2 degrees 2-theta; a solid state ¹³C NMR spectrum having peaks at 44.8, 51.7, 135.4, 147.5, 150.2 and 154.4 ppm±0.2 ppm; a solid state ¹³C NMR spectrum having the following chemical shift absolute differences from a reference peak at 102.6 ppm±2 ppm of 57.8, 50.9, 32.8, 44.9, 47.6 and 51.8 ppm±0.1 ppm; a solid state ¹³C NMR spectrum substantially as depicted in FIG. 21a, 21b or 21c; and combinations of these data. Optionally, crystalline Form PL11 of Paltusotine according to any aspect or embodiment of the disclosure may be characterized by an X-ray powder diffraction pattern substantially as depicted in FIG. 2; an X-ray powder diffraction pattern having peaks at 4.5, 7.1, 15.2, 18.6 and 21.7 degrees 2-theta±0.2 degrees 2-theta; and an absence of a peak at 12.0 degrees 2-theta±0.2 degrees 2-theta.

Crystalline Form PL11 of Paltusotine may be further characterized by an X-ray powder diffraction pattern having peaks at 4.5, 7.1, 15.2, 18.6 and 21.7 degrees 2-theta±0.2 degrees 2-theta, and also having any one, two, three, or four additional peaks selected from 11.4, 18.2, 28.1 and 30.7 degrees 2-theta±0.2 degrees 2-theta. Alternatively, crystalline Form PL11 of Paltusotine according to any aspect or embodiment of the disclosure may be characterized by an X-ray powder diffraction pattern having peaks at 4.5, 7.1, 15.2, 18.6 and 21.7 degrees 2-theta±0.2 degrees 2-theta, and also having any one, two, three, or four additional peaks selected from 11.4, 18.2, 28.1 and 30.7 degrees 2-theta±0.2 degrees 2-theta; and an absence of a peak at 12.0 degrees 2-theta±0.2 degrees 2-theta.

Alternatively crystalline Form PL11 of Paltusotine according to any aspect or embodiment of the present disclosure may be characterized by an X-ray powder diffraction pattern having peaks at 4.5, 7.1, 15.2, 18.6 and 21.7 degrees 2-theta±0.2 degrees 2-theta, and also having any one additional peak selected from 11.4, 18.2, 28.1 and 30.7 degrees 2-theta±0.2 degrees 2-theta; and optionally an absence of a peak at 12.0 degrees 2-theta±0.2 degrees 2-theta. Crystalline Form PL11 of Paltusotine according to any aspect or embodiment of the present disclosure may alternatively be characterized by an X-ray powder diffraction pattern having peaks at 4.5, 7.1, 15.2, 18.6 and 21.7 degrees 2-theta±0.2 degrees 2-theta, and also having two additional peaks selected from 11.4, 18.2, 28.1 and 30.7 degrees 2-theta±0.2 degrees 2-theta; and optionally an absence of a peak at 12.0 degrees 2-theta±0.2 degrees 2-theta.

Crystalline Form PL11 of Paltusotine may be alternatively characterized by an X-ray powder diffraction pattern having peaks at 4.5, 7.1, 11.4, 15.2, 18.2, 18.6, 21.7, 28.1 and 30.7 degrees 2-theta±0.2 degrees 2-theta; and optionally an absence of a peak at 12.0 degrees 2-theta±0.2 degrees 2-theta.

In one embodiment of the present disclosure, crystalline Form PL11 of Paltusotine is isolated.

Crystalline Form PL11 of Paltusotine may be hydrate form, more preferably monohydrate. Crystalline Form PL11 of Paltusotine according to any aspect or embodiment of the disclosure may optionally contain from about 3.0 to about 6.0% by weight of water, measured, for example by Karl Fischer analysis, or by TGA.

Crystalline Form PL11 of Paltusotine may be characterized by each of the above characteristics alone/or by all possible combinations, e.g., an XRPD pattern having peaks at 4.5, 7.1, 15.2, 18.6 and 21.7 degrees 2-theta±0.2 degrees 2-theta; an XRPD pattern as depicted in FIG. 2, and combinations thereof.

The present disclosure further comprises a process for preparation of Form PL11 of Paltusotine. Thus, this process may comprise:
(i) suspending Paltusotine Pattern A in buffer solution;
(ii) stirring the suspension;
(iii) filtering the solid;
(iv) suspending the wet solid in water;
(v) optionally isolating Form PL11 of Paltusotine; and
(vi) optionally drying Form PL11 of Paltusotine.

In any embodiment of this process, the pH of buffer solution used in step (i) is 6.8. Particularly, the buffer solution is a phosphate buffer having a pH of 6.8. Such a buffer solution may be prepared according to any suitable method, particularly in accordance with the US Pharmacopoeia (USP 35-NF 30 Buffer solutions). Particularly, the buffer solution may be prepared as described in the examples below.

According to any aspect or embodiment of the process, the buffer solution may be used in an amount of about 5 ml to about 15 ml, about 8 ml to about 12 ml, or about 10 ml, per gram of Paltusotine Pattern A. Step (ii) comprises stirring the suspension for period of about 15 hour to 60 hours, or about 24 hours to about 48 hours. Step (iii) comprises filtering the solid which may be carried out at a temperature of at the temperature of about 10° C. to about 40° C., about 15° C. to about 30° C., or about 25° C. Step (iv) comprises suspending the wet solid in water. According to any aspect or embodiment of the process, the water may be used in an amount of about 5 ml to about 15 ml, about 8 ml to about 12 ml, or about 10 ml, per gram of Paltusotine Pattern A.

The PL11 Form of Paltusotine may be isolated, preferably by any suitable process, such as decantation, filtration or by centrifuge, preferably by filtration under vacuum. The filtering may be carried out at a temperature of at the temperature of about 10° C. to about 40° C., about 15° C. to about 30° C., or about 25° C.

In any embodiment of this process, the crystalline Paltusotine Form PL11 may be dried under vacuum. The crystalline Paltusotine Form PL11 may be dried under vacuum, typically at a temperature of about 40° C. to about 80° C., about 50° C. to about 70° C., about 60° C. The drying may be carried out for any suitable time to remove the solvent, typically about 1 to about 5 hours, about 1.5 hours to about 4 hours, or about 2 hours.

The present disclosure further comprises a process for preparation of Form PL11 of Paltusotine. Thus, this process may comprise:
(i) suspending Paltusotine Form PL1 in methyl t-butyl ether (MTBE);
(ii) stirring the suspension;
(iii) isolating Form PL11 of Paltusotine; and
(iv) drying Form PL11 of Paltusotine.

The Paltusotine Form PL1 may be suspended in MTBE at temperature of about 20° C. to about 60° C., about 10° C. to about 50° C., about 20° C. to about 40° C., or about 25° C. According to any aspect or embodiment of the process, the MTBE may be used in an amount of about 10 ml to about 80 ml, about 15 ml to about 70 ml, or about 40 ml, per gram of Paltusotine Form PL1. Step (ii) comprises stirring the suspension for period of about 15 hour to 60 hours, or about 24 hours to about 48 hours. The PL11 Form of Paltusotine may be isolated, preferably by any suitable process, such as decantation, filtration or by centrifuge, preferably by filtration under vacuum. The filtering may be carried out at a temperature of at the temperature of about 10° C. to about 40° C., about 15° C. to about 30° C., or about 25° C.

In any embodiment of this process, the crystalline Paltusotine Form PL11 may be dried under vacuum. The crystalline Form PL11 of Paltusotine may be dried under vacuum, typically at a temperature of about 40° C. to about 80° C., about 50° C. to about 70° C., about 60° C. The drying may be carried out for any suitable time to remove the solvent, typically about 1 to about 5 hours, about 1.5 hours to about 4 hours, or about 2 hours.

In accordance with any embodiment of the process, form PL11 of Paltusotine may be prepared by a process comprising:
(i) providing a solution of Paltusotine in acetic acid;
(ii) combining the solution with ethyl acetate;
(iii) maintaining the solution for suitable period of time;
(iv) filtering the solid;
(v) suspending the wet solid in water;
(vi) optionally isolating Form PL11 of Paltusotine; and
(vii) optionally drying the Form PL11 of Paltusotine.

In any embodiment of this process, the solution in step (i) may be prepared by dissolving Paltusotine in acetic acid. The acetic acid may be dissolved at a temperature of about 20° C. to about 60° C., about 22° C. to about 50° C., about 24° C. to about 40° C., or about 25° C. According to any aspect or embodiment of the process, the acetic acid may be used in an amount of about 5 ml to about 30 ml, about 8 ml to about 20 ml, or about 10 ml, per gram of Paltusotine. Step (ii) may comprise combining the solution of step (i) with ethyl acetate. Preferably step (ii) comprises combining the solution of step (i) with ethyl acetate at a temperature of: 20° C. to about 60° C., about 22° C. to about 50° C., about 24° C. to about 40° C., or about 25° C. for period of about 3 hour to 1 hour, or about 2 hours. According to any aspect or embodiment of the process, the ethyl acetate may be used in an amount of about 20 ml to about 80 ml, about 20 ml to about 70 ml, or about 67 ml, per gram of Paltusotine.

Step (iii) comprises filtering the solid which may be carried out at a temperature of at the temperature of about 10° C. to about 40° C., about 15° C. to about 30° C., or about 25° C. Step (iv) comprises suspending the wet solid in water. According to any aspect or embodiment of the process, the water may be used in an amount of about 33.3 ml, per gram of Paltusotine.

The PL11 Form of Paltusotine may be isolated, preferably by any suitable process, such as decantation, filtration or by centrifuge, preferably by filtration under vacuum. The filtering may be carried out at a temperature of at the temperature of about 10° C. to about 40° C., about 15° C. to about 30° C., or about 25° C.

In any embodiment of this process, the crystalline Paltusotine Form PL11 may be dried under vacuum. The crystalline Paltusotine Form PL11 may be dried under vacuum, typically at a temperature of about 40° C. to about 80° C., about 50° C. to about 70° C., about 60° C. The drying may be carried out for any suitable time to remove the solvent, typically about 1 to about 5 hours, about 1.5 hours to about 4 hours, or about 2 hours.

According to any aspect or embodiment of the processes for preparing Paltusotine Form PL11, the process may further comprise combining the Paltusotine Form PL11 with at least one pharmaceutically acceptable excipient to prepare a pharmaceutical composition or dosage form.

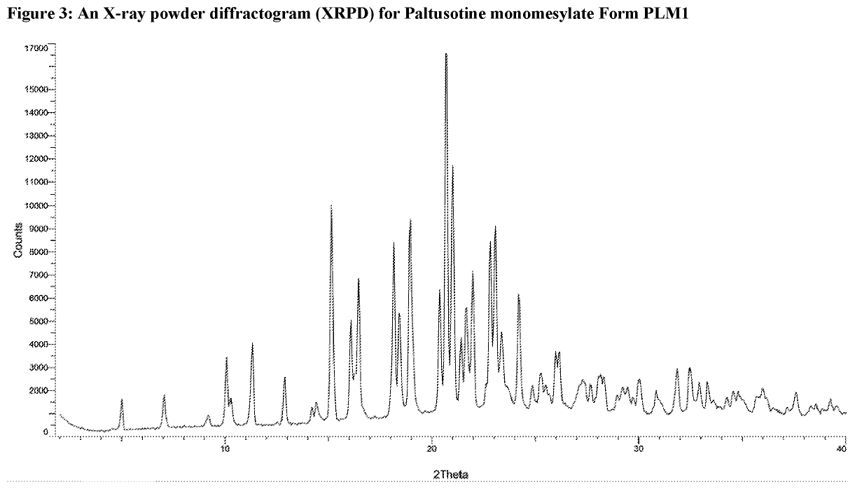

The present disclosure includes a crystalline polymorph of Paltusotine monomesylate, designated PLM1. The crystalline Form PLM1 of Paltusotine monomesylate may be characterized by data selected from one or more of the following: an X-ray powder diffraction pattern substantially as depicted in FIG. 3; an X-ray powder diffraction pattern having peaks at 10.1, 11.4, 15.2, 16.5 and 24.2 degrees 2-theta±0.2 degrees 2-theta; and combinations of these data.

Crystalline Form PLM1 of Paltusotine monomesylate may be further characterized by an X-ray powder diffraction pattern having peaks at 10.1, 11.4, 15.2, 16.5 and 24.2 degrees 2-theta±0.2 degrees 2-theta, and also having any one, two, or three additional peaks selected from 16.1, 18.2, 26.2 and 32.6 degrees 2-theta±0.2 degrees 2-theta.

Crystalline Form PLM1 of Paltusotine monomesylate may be alternatively characterized by an X-ray powder diffraction pattern having peaks at 10.1, 11.4, 15.2, 16.1, 16.5, 18.2, 24.2, 26.2 and 32.6 degrees 2-theta±0.2 degrees 2-theta.

In one embodiment of the present disclosure, crystalline Form PLM1 of Paltusotine monomesylate is isolated.

Crystalline Form PLM1 of Paltusotine monomesylate may be anhydrous.

Crystalline Form PLM1 of Paltusotine monomesylate may be characterized by each of the above characteristics alone/or by all possible combinations, e.g., an XRPD pattern having peaks at 10.1, 11.4, 15.2, 16.5 and 24.2 degrees 2-theta±0.2 degrees 2-theta; an XRPD pattern as depicted in FIG. 3, and combinations thereof.

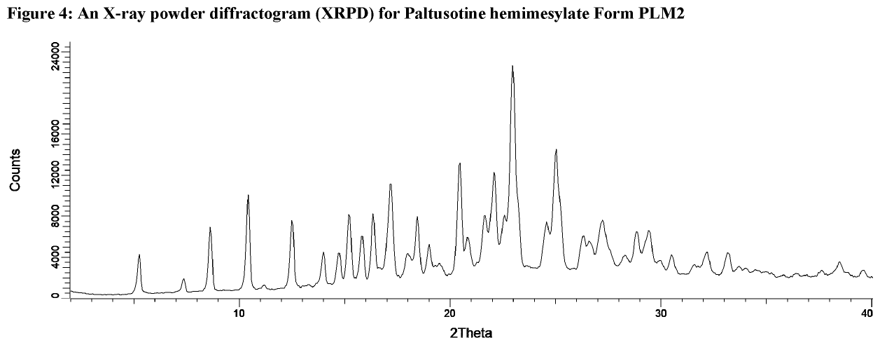

The present disclosure includes a crystalline polymorph of Paltusotine hemimesylate, designated PLM2. The crystalline Form PLM2 of Paltusotine hemimesylate may be characterized by data selected from one or more of the following: an X-ray powder diffraction pattern substantially as depicted in FIG. 4; an X-ray powder diffraction pattern having peaks at 8.6, 10.4, 12.5, 15.2 and 23.0 degrees 2-theta±0.2 degrees 2-theta; and combinations of these data.

Crystalline Form PLM2 of Paltusotine hemimesylate may be further characterized by an X-ray powder diffraction pattern having peaks at 8.6, 10.4, 12.5, 15.2 and 23.0 degrees 2-theta±0.2 degrees 2-theta, and also having any one, two, three, or four additional peaks selected from 16.4, 20.4, 27.2 and 28.9 degrees 2-theta±0.2 degrees 2-theta.

Crystalline Form PLM2 of Paltusotine hemimesylate may be alternatively characterized by an X-ray powder diffraction pattern having peaks at 8.6, 10.4, 12.5, 15.2, 16.4, 20.4, 23.0, 27.2 and 28.9 degrees 2-theta±0.2 degrees 2-theta.

In one embodiment of the present disclosure, crystalline Form PLM2 of Paltusotine hemimesylate is isolated.

Crystalline Form PLM2 of Paltusotine hemimesylate may be anhydrous.

Crystalline Form PLM2 of Paltusotine hemimesylate may be characterized by each of the above characteristics alone/or by all possible combinations, e.g., an XRPD pattern having peaks at 8.6, 10.4, 12.5, 15.2 and 23.0 degrees 2-theta±0.2 degrees 2-theta; an XRPD pattern as depicted in FIG. 4, and combinations thereof.

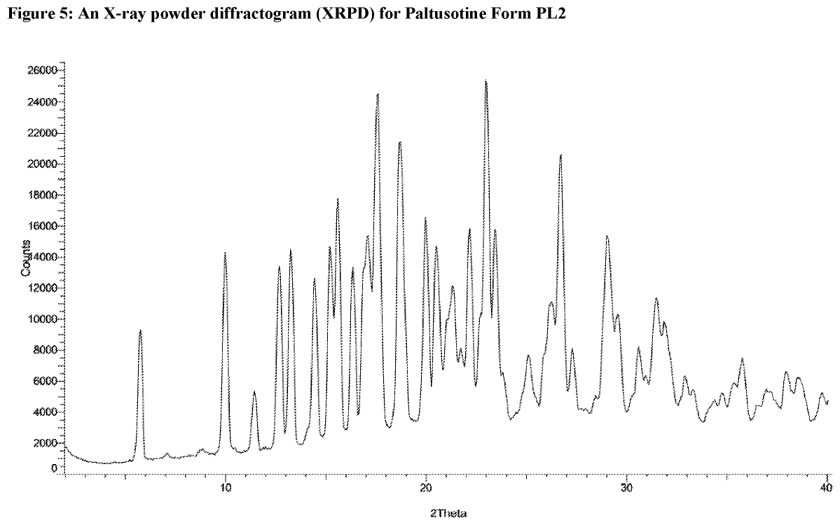

The present disclosure includes a crystalline polymorph of Paltusotine, designated PL2. The crystalline Form PL2 of Paltusotine may be characterized by data selected from one or more of the following: an X-ray powder diffraction pattern substantially as depicted in FIG. 5; an X-ray powder diffraction pattern having peaks at 5.7, 10.0, 12.7, 18.7 and 20.5 degrees 2-theta±0.2 degrees 2-theta; and combinations of these data.

Crystalline Form PL2 of Paltusotine may be further characterized by an X-ray powder diffraction pattern having peaks at 5.7, 10.0, 12.7, 18.7 and 20.5 degrees 2-theta±0.2 degrees 2-theta, and also having any one, two, three, or four additional peaks selected from 15.2, 15.6, 23.4 and 29.0 degrees 2-theta±0.2 degrees 2-theta.

Crystalline Form PL2 of Paltusotine may be alternatively characterized by an X-ray powder diffraction pattern having peaks at 5.7, 10.0, 12.7, 15.2, 15.6, 18.7 20.5, 23.4 and 29.0 degrees 2-theta±0.2 degrees 2-theta.

In one embodiment of the present disclosure, crystalline Form PL2 of Paltusotine is isolated.

Crystalline Form PL2 of Paltusotine may be acetonitrile solvate.

Crystalline Form PL2 of Paltusotine may be characterized by each of the above characteristics alone/or by all possible combinations, e.g., an XRPD pattern having peaks at 5.7, 10.0, 12.7, 18.7 and 20.5 degrees 2-theta±0.2 degrees 2-theta; an XRPD pattern as depicted in FIG. 5, and combinations thereof.

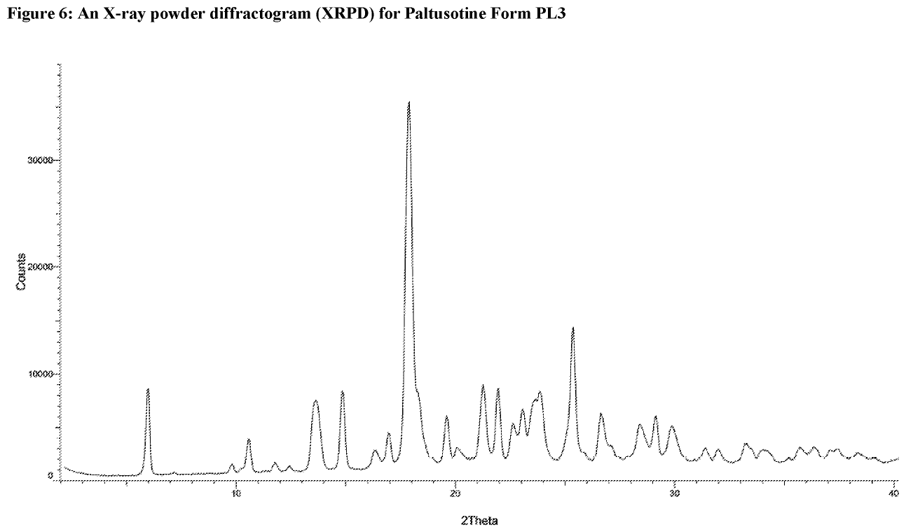

The present disclosure includes a crystalline polymorph of Paltusotine, designated PL3. The crystalline Form PL3 of Paltusotine may be characterized by data selected from one or more of the following: an X-ray powder diffraction pattern substantially as depicted in FIG. 6; an X-ray powder diffraction pattern having peaks at 6.0, 10.6, 18.1, 19.5 and 25.3 degrees 2-theta±0.2 degrees 2-theta; and combinations of these data.

Crystalline Form PL3 of Paltusotine may be further characterized by an X-ray powder diffraction pattern having peaks at 6.0, 10.6, 18.1, 19.5 and 25.3 degrees 2-theta±0.2 degrees 2-theta, and also having any one, two, three or four additional peaks selected from 14.8, 21.2, 21.9 and 23.8 degrees 2-theta±0.2 degrees 2-theta.

Crystalline Form PL3 of Paltusotine may be alternatively characterized by an X-ray powder diffraction pattern having peaks at 6.0, 10.6, 14.8, 18.1, 19.5, 21.2, 21.9, 23.8 and 25.3 degrees 2-theta±0.2 degrees 2-theta.

In one embodiment of the present disclosure, crystalline Form PL3 of Paltusotine is isolated.

Crystalline Form PL3 of Paltusotine may be 1-butanol solvate.

Crystalline Form PL3 of Paltusotine may be characterized by each of the above characteristics alone/or by all possible combinations, e.g., an XRPD pattern having peaks at 6.0, 10.6, 18.1, 19.5 and 25.3 degrees 2-theta±0.2 degrees 2-theta; an XRPD pattern as depicted in FIG. 6, and combinations thereof.

The present disclosure includes a crystalline polymorph of Paltusotine, designated PL4. The crystalline Form PL4 of Paltusotine may be characterized by data selected from one or more of the following: an X-ray powder diffraction pattern substantially as depicted in FIG. 7; an X-ray powder diffraction pattern having peaks at 11.7, 15.4, 17.3, 19.7 and 23.1 degrees 2-theta±0.2 degrees 2-theta; and combinations of these data.

Crystalline Form PL4 of Paltusotine may be further characterized by an X-ray powder diffraction pattern having peaks at 11.7, 15.4, 17.3, 19.7 and 23.1 degrees 2-theta±0.2 degrees 2-theta, and also having any one, two, three or four additional peaks selected from 7.3, 8.3, 17.9 and 23.6 degrees 2-theta±0.2 degrees 2-theta.

Crystalline Form PL4 of Paltusotine may be alternatively characterized by an X-ray powder diffraction pattern having peaks at 7.3, 8.3, 11.7, 15.4, 17.3, 17.9, 19.7, 23.1 and 23.6 degrees 2-theta±0.2 degrees 2-theta.

In one embodiment of the present disclosure, crystalline Form PL4 of Paltusotine is isolated.

Crystalline Form PL4 of Paltusotine may be diethyl ketone solvate form.

Crystalline Form PL4 of Paltusotine may be characterized by each of the above characteristics alone/or by all possible combinations, e.g., an XRPD pattern having peaks at 11.7, 15.4, 17.3, 19.7 and 23.1 degrees 2-theta±0.2 degrees 2-theta; an XRPD pattern as depicted in FIG. 7, and combinations thereof.

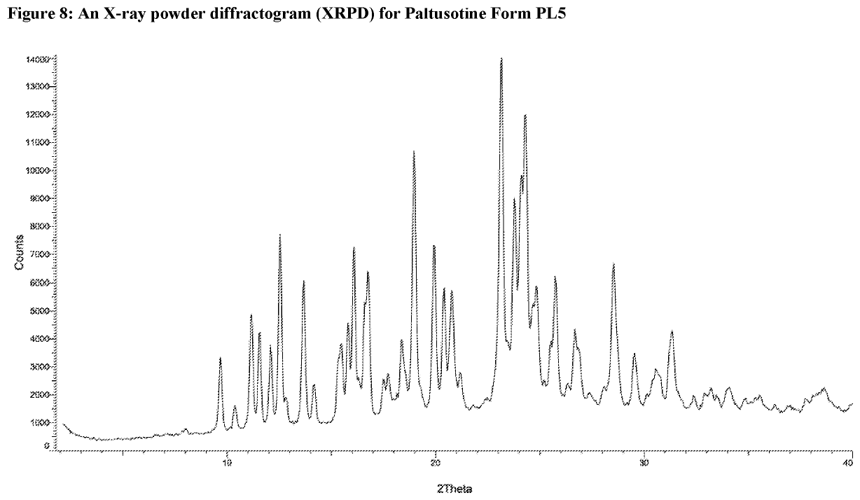

The present disclosure includes a crystalline polymorph of Paltusotine, designated PL5. The crystalline Form PL5 of Paltusotine may be characterized by data selected from one or more of the following: an X-ray powder diffraction pattern substantially as depicted in FIG. 8; an X-ray powder diffraction pattern having peaks at 11.2, 12.5, 13.7, 19.9 and 24.3 degrees 2-theta±0.2 degrees 2-theta; and combinations of these data.

Crystalline Form PL5 of Paltusotine may be further characterized by an X-ray powder diffraction pattern having peaks at 11.2, 12.5, 13.7, 19.9 and 24.3 degrees 2-theta±0.2 degrees 2-theta, and also having any one, two, three, or four additional peaks selected from 11.5, 14.1, 25.7 and 31.3 degrees 2-theta±0.2 degrees 2-theta.

Crystalline Form PL5 of Paltusotine may be alternatively characterized by an X-ray powder diffraction pattern having peaks at 11.2, 11.5, 12.5, 13.7, 14.1, 19.9, 24.3, 25.7 and 31.3 degrees 2-theta±0.2 degrees 2-theta.

In one embodiment of the present disclosure, crystalline Form PL5 of Paltusotine is isolated.

Crystalline Form PL5 of Paltusotine may be 1, 2-dimethoxyethane solvate.

Crystalline Form PL5 of Paltusotine may be characterized by each of the above characteristics alone/or by all possible combinations, e.g., an XRPD pattern having peaks at 11.2, 12.5, 13.7, 19.9 and 24.3 degrees 2-theta±0.2 degrees 2-theta; an XRPD pattern as depicted in FIG. 8, and combinations thereof.

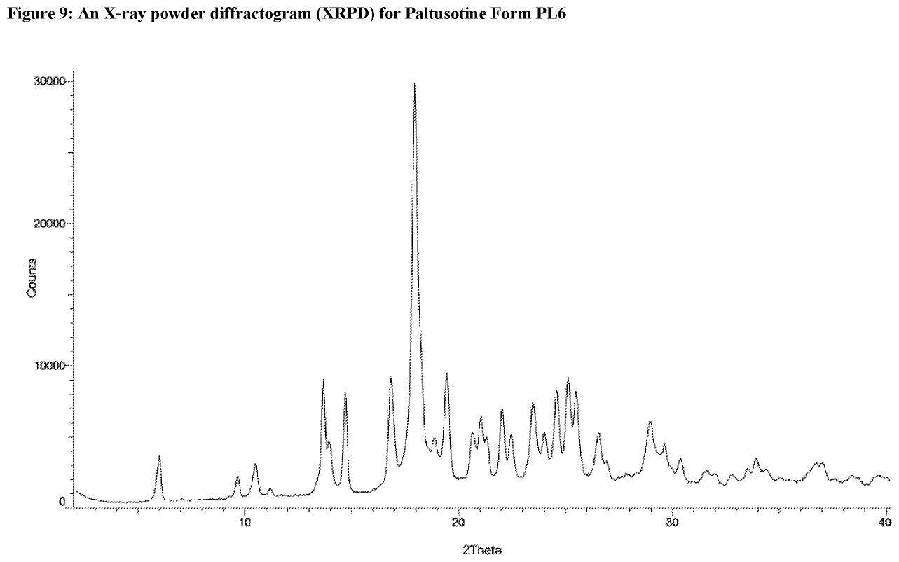

The present disclosure includes a crystalline polymorph of Paltusotine, designated PL6. The crystalline Form PL6 of Paltusotine may be characterized by data selected from one or more of the following: an X-ray powder diffraction pattern substantially as depicted in FIG. 9; an X-ray powder diffraction pattern having peaks at 5.4, 14.8, 15.8, 19.2 and 20.3 degrees 2-theta±0.2 degrees 2-theta; and combinations of these data.

Crystalline Form PL6 of Paltusotine may be further characterized by an X-ray powder diffraction pattern having peaks at 5.4, 14.8, 15.8, 19.2 and 20.3 degrees 2-theta±0.2 degrees 2-theta, and also having any one, two, three, or four additional peaks selected from 13.7, 22.5, 26.6 and 29.9 degrees 2-theta±0.2 degrees 2-theta.

Crystalline Form PL6 of Paltusotine may be alternatively characterized by an X-ray powder diffraction pattern having peaks at 5.4, 13.7, 14.8, 15.8, 19.2, 20.3, 22.5, 26.6 and 29.9 degrees 2-theta±0.2 degrees 2-theta.

In one embodiment of the present disclosure, crystalline Form PL6 of Paltusotine is isolated.

Crystalline Form PL6 of Paltusotine may be crystalline hexanol solvate.

Crystalline Form PL6 of Paltusotine may be characterized by each of the above characteristics alone/or by all possible combinations, e.g., an XRPD pattern having peaks at 5.4, 14.8, 15.8, 19.2 and 20.3 degrees 2-theta±0.2 degrees 2-theta; an XRPD pattern as depicted in FIG. 9, and combinations thereof.

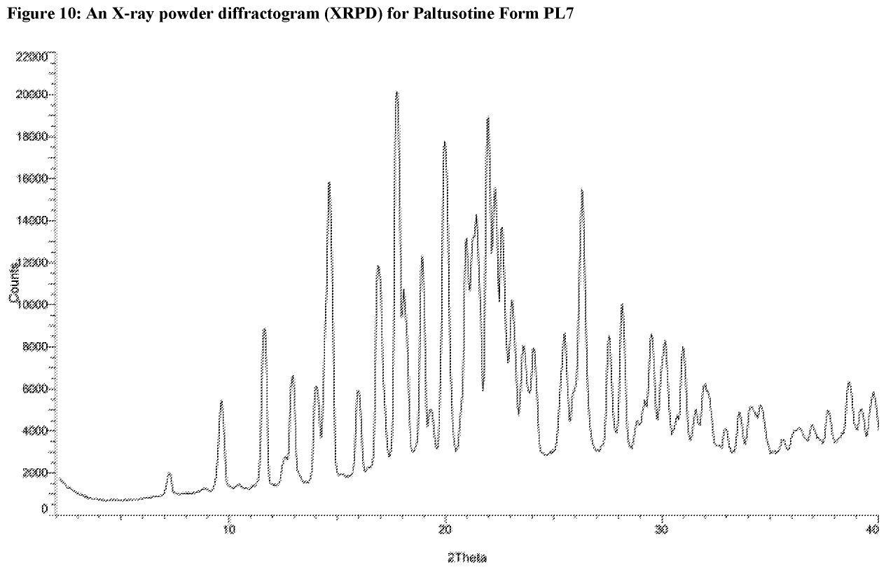

The present disclosure includes a crystalline polymorph of Paltusotine, designated PL7. The crystalline Form PL7 of Paltusotine may be characterized by data selected from one or more of the following: an X-ray powder diffraction pattern substantially as depicted in FIG. 10; an X-ray powder diffraction pattern having peaks at 9.7, 12.9, 16.9, 18.9 and 27.6 degrees 2-theta±0.2 degrees 2-theta; and combinations of these data.

Crystalline Form PL7 of Paltusotine may be further characterized by an X-ray powder diffraction pattern having peaks at 9.7, 12.9, 16.9, 18.9 and 27.6 degrees 2-theta±0.2 degrees 2-theta, and also having any one, two, three or four additional peaks selected from 11.6, 17.8, 20.0 and 22.6 degrees 2-theta±0.2 degrees 2-theta.

Crystalline Form PL7 of Paltusotine may be alternatively characterized by an X-ray powder diffraction pattern having peaks at 9.7, 11.6, 12.9, 16.9, 17.8, 18.9 20.0, 22.6 and 27.6 degrees 2-theta±0.2 degrees 2-theta.

In one embodiment of the present disclosure, crystalline Form PL7 of Paltusotine is isolated.

Crystalline Form PL7 of Paltusotine may be methyl acetate solvate.

Crystalline Form PL7 of Paltusotine may be characterized by each of the above characteristics alone/or by all possible combinations, e.g., an XRPD pattern having peaks at 9.7, 12.9, 16.9, 18.9 and 27.6 degrees 2-theta±0.2 degrees 2-theta; an XRPD pattern as depicted in FIG. 10, and combinations thereof.

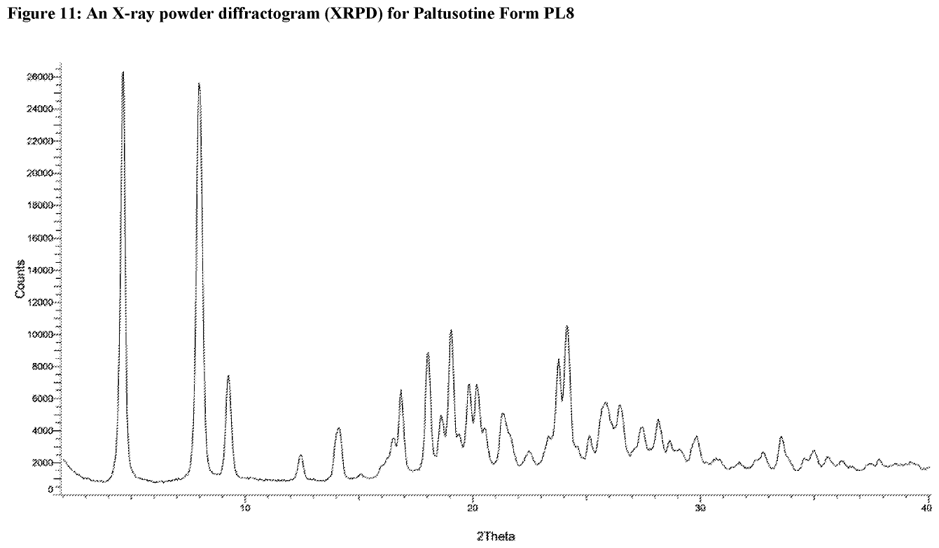

The present disclosure includes a crystalline polymorph of Paltusotine, designated PL8. The crystalline Form PL8 of Paltusotine may be characterized by data selected from one or more of the following: an X-ray powder diffraction pattern substantially as depicted in FIG. 11; an X-ray powder diffraction pattern having peaks at 4.6, 7.9, 9.2, 20.2 and 26.5 degrees 2-theta±0.2 degrees 2-theta; and combinations of these data.

Crystalline Form PL8 of Paltusotine may be further characterized by an X-ray powder diffraction pattern having peaks at 4.6, 7.9, 9.2, 20.2 and 26.5 degrees 2-theta±0.2 degrees 2-theta, and also having any one, two, three or four additional peaks selected from 12.4, 14.1, 19.0 and 21.3 degrees 2-theta±0.2 degrees 2-theta.

Crystalline Form PL8 of Paltusotine may be alternatively characterized by an X-ray powder diffraction pattern having peaks at 4.6, 7.9, 9.2, 12.4, 14.1, 19.0, 20.2, 21.3 and 26.5 degrees 2-theta±0.2 degrees 2-theta.

In one embodiment of the present disclosure, crystalline Form PL8 of Paltusotine is isolated.

Crystalline Form PL8 of Paltusotine may be tert-amyl alcohol solvate.

Crystalline Form PL8 of Paltusotine may be characterized by each of the above characteristics alone/or by all possible combinations, e.g., an XRPD pattern having peaks at 4.6, 7.9, 9.2, 20.2 and 26.5 degrees 2-theta±0.2 degrees 2-theta; an XRPD pattern as depicted in FIG. 11, and combinations thereof.

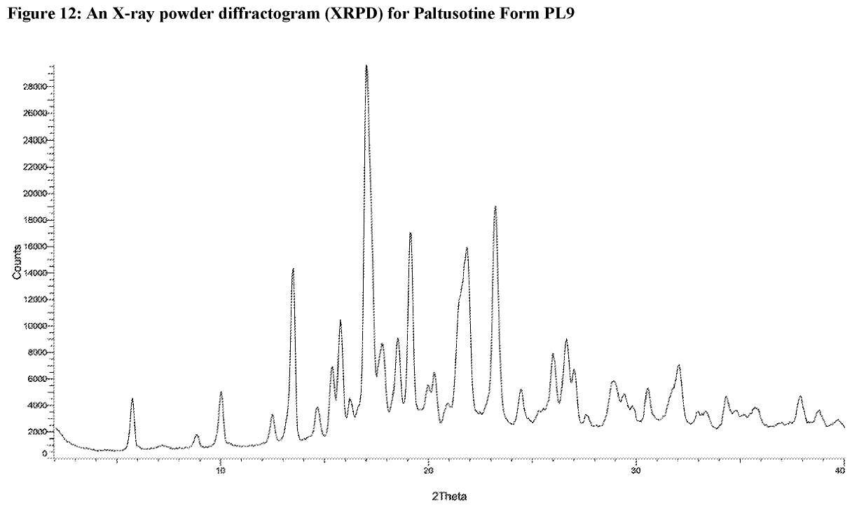

The present disclosure includes a crystalline polymorph of Paltusotine, designated PL9. The crystalline Form PL9 of Paltusotine may be characterized by data selected from one or more of the following: an X-ray powder diffraction pattern substantially as depicted in FIG. 12; an X-ray powder diffraction pattern having peaks at 13.4, 16.2, 23.2, 26.0 and 32.0 degrees 2-theta±0.2 degrees 2-theta; and combinations of these data.

Crystalline Form PL9 of Paltusotine may be further characterized by an X-ray powder diffraction pattern having peaks at 13.4, 16.2, 23.2, 26.0 and 32.0 degrees 2-theta±0.2 degrees 2-theta, and also having any one, two, three, or four additional peaks selected from 8.8, 17.1, 21.9 and 28.9 degrees 2-theta±0.2 degrees 2-theta.

Crystalline Form PL9 of Paltusotine may be alternatively characterized by an X-ray powder diffraction pattern having peaks at 8.8, 13.4, 16.2, 17.1, 21.9, 23.2, 26.0, 28.9 and 32.0 degrees 2-theta±0.2 degrees 2-theta.

In one embodiment of the present disclosure, crystalline Form PL9 of Paltusotine is isolated.

Crystalline Form PL9 of Paltusotine may be 1,4-Dioxane solvate.

Crystalline Form PL9 of Paltusotine may be characterized by each of the above characteristics alone/or by all possible combinations, e.g., an XRPD pattern having peaks at 13.4, 16.2, 23.2, 26.0 and 32.0 degrees 2-theta±0.2 degrees 2-theta; an XRPD pattern as depicted in FIG. 12, and combinations thereof.

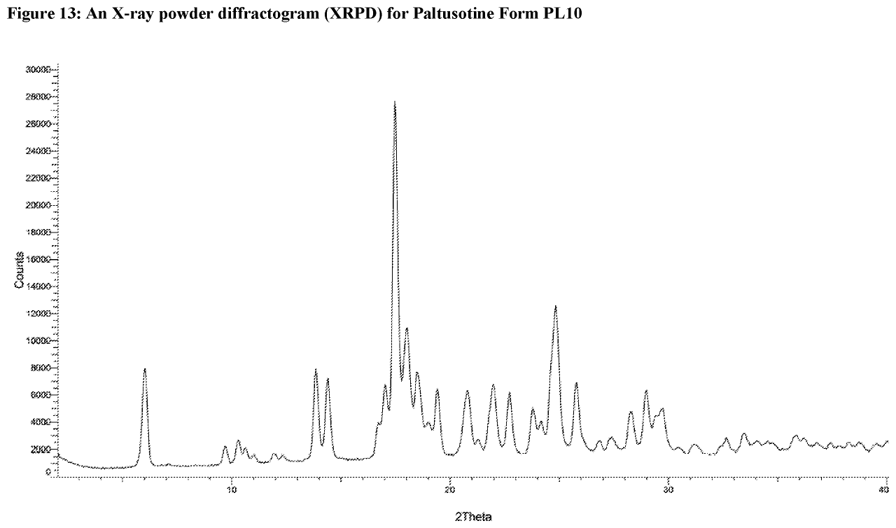

The present disclosure includes a crystalline polymorph of Paltusotine, designated PL10. The crystalline Form PL10 of Paltusotine may be characterized by data selected from one or more of the following: an X-ray powder diffraction pattern substantially as depicted in FIG. 13; an X-ray powder diffraction pattern having peaks at 6.0, 17.5, 20.8, 22.7, 24.8 and 25.8 degrees 2-theta±0.2 degrees 2-theta; and combinations of these data.

Crystalline Form PL10 of Paltusotine may be further characterized by an X-ray powder diffraction pattern having peaks at 6.0, 17.5, 20.8, 22.7, 24.8 and 25.8 degrees 2-theta±0.2 degrees 2-theta, and also having any one, two, three, four or five additional peaks selected from 13.9, 14.4, 18.5 and 29.0 degrees 2-theta±0.2 degrees 2-theta.

Crystalline Form PL10 of Paltusotine may be alternatively characterized by an X-ray powder diffraction pattern having peaks at 6.0, 13.9, 14.4, 17.5, 18.5, 20.8, 22.7, 24.8, 25.8 and 29.0 degrees 2-theta±0.2 degrees 2-theta.

In one embodiment of the present disclosure, crystalline Form PL10 of Paltusotine is isolated.

Crystalline Form PL10 of Paltusotine may be Isobutanol solvate.

Crystalline Form PL10 of Paltusotine may be characterized by each of the above characteristics alone/or by all possible combinations, e.g., an XRPD pattern having peaks at 6.0, 17.5, 20.8, 22.7, 24.8 and 25.8 degrees 2-theta±0.2 degrees 2-theta; an XRPD pattern as depicted in FIG. 13, and combinations thereof.

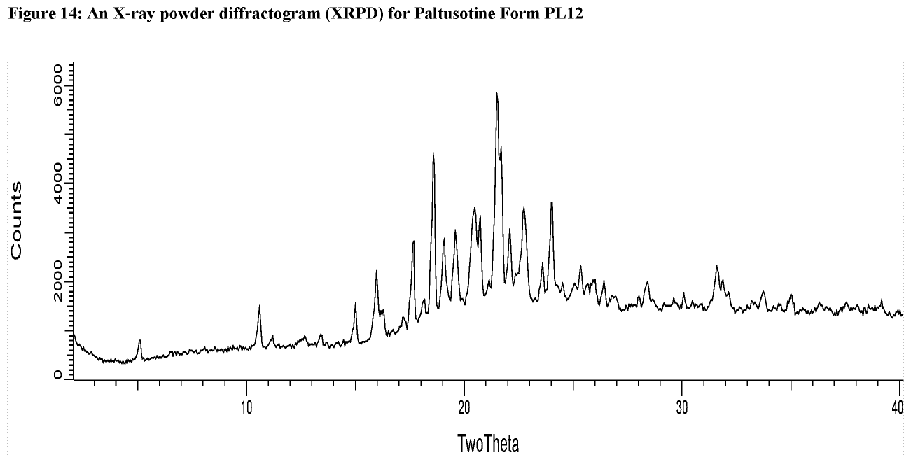

The present disclosure includes a crystalline polymorph of Paltusotine, designated PL12. The crystalline Form PL12 of Paltusotine may be characterized by data selected from one or more of the following: an X-ray powder diffraction pattern substantially as depicted in FIG. 14; an X-ray powder diffraction pattern having peaks at 10.5, 17.6, 18.5, 19.0 and 19.5 degrees 2-theta±0.2 degrees 2-theta; and combinations of these data.

Crystalline Form PL12 of Paltusotine may be further characterized by an X-ray powder diffraction pattern having peaks at 10.5, 17.6, 18.5, 19.0 and 19.5 degrees 2-theta±0.2 degrees 2-theta, and also having any one, two, three or four additional peaks selected from 15.9, 21.5, 22.7 and 24.0 degrees 2-theta±0.2 degrees 2-theta.

Crystalline Form PL12 of Paltusotine may be alternatively characterized by an X-ray powder diffraction pattern having peaks at 10.5, 15.9, 17.6, 18.5, 19.0, 19.5, 21.5, 22.7 and 24.0 degrees 2-theta±0.2 degrees 2-theta.

In one embodiment of the present disclosure, crystalline Form PL12 of Paltusotine is isolated.

Crystalline Form PL12 of Paltusotine may be characterized by each of the above characteristics alone/or by all possible combinations, e.g., an XRPD pattern having peaks at 10.5, 17.6, 18.5, 19.0 and 19.5 degrees 2-theta±0.2 degrees 2-theta; an XRPD pattern as depicted in FIG. 14, and combinations thereof.

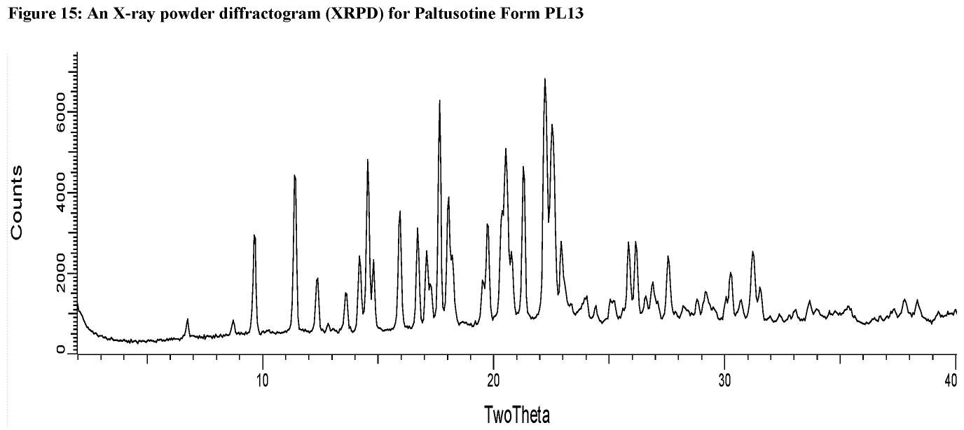

The present disclosure includes a crystalline polymorph of Paltusotine, designated PL13. The crystalline Form PL13 of Paltusotine may be characterized by data selected from one or more of the following: an X-ray powder diffraction pattern substantially as depicted in FIG. 15; an X-ray powder diffraction pattern having peaks at 9.8, 11.6, 16.1, 17.9 and 21.5 degrees 2-theta±0.2 degrees 2-theta; and combinations of these data.

Crystalline Form PL13 of Paltusotine may be further characterized by an X-ray powder diffraction pattern having peaks at 9.8, 11.6, 16.1, 17.9 and 21.5 degrees 2-theta±0.2 degrees 2-theta, and also having any one, two, three or four additional peaks selected from 12.6, 14.8, 20.7 and 22.4 degrees 2-theta±0.2 degrees 2-theta.

Crystalline Form PL13 of Paltusotine may be alternatively characterized by an X-ray powder diffraction pattern having peaks at 9.8, 11.6, 12.6, 14.8, 16.1, 17.9, 20.7, 21.5 and 22.4 degrees 2-theta±0.2 degrees 2-theta.

In one embodiment of the present disclosure, crystalline Form PL13 of Paltusotine is isolated.

Crystalline Form PL13 of Paltusotine may be characterized by each of the above characteristics alone/or by all possible combinations, e.g., an XRPD pattern having peaks at 9.8, 11.6, 16.1, 17.9 and 21.5 degrees 2-theta±0.2 degrees 2-theta; an XRPD pattern as depicted in FIG. 15, and combinations thereof.

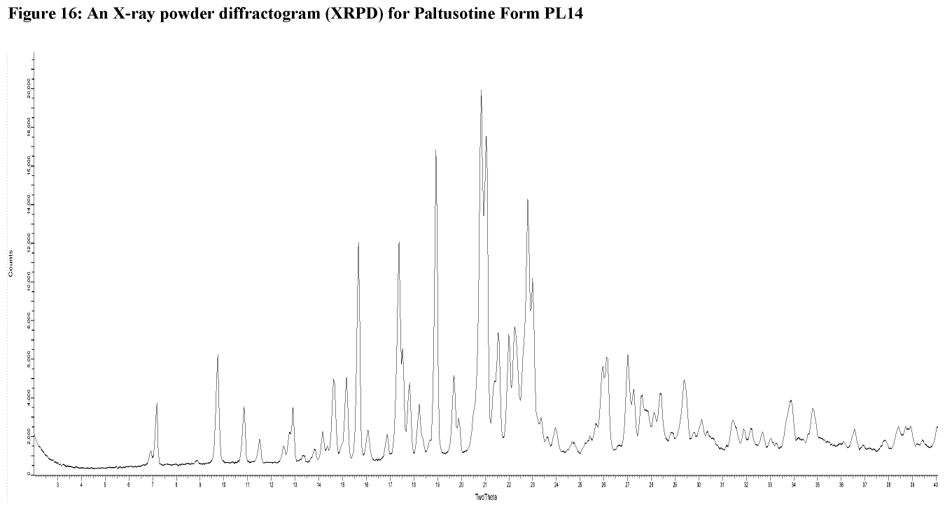

The present disclosure includes a crystalline polymorph of Paltusotine, designated PL14. The crystalline Form PL14 of Paltusotine may be characterized by data selected from one or more of the following: an X-ray powder diffraction pattern substantially as depicted in FIG. 16; an X-ray powder diffraction pattern having peaks at 9.7, 10.8, 15.6, 18.9 and 29.3 degrees 2-theta±0.2 degrees 2-theta; and combinations of these data.

Crystalline Form PL14 of Paltusotine may be further characterized by an X-ray powder diffraction pattern having peaks at 9.7, 10.8, 15.6, 18.9 and 29.3 degrees 2-theta±0.2 degrees 2-theta, and also having any one, two, three, or four additional peaks selected from 17.3, 19.6 and 22.8 degrees 2-theta±0.2 degrees 2-theta.

Crystalline Form PL14 of Paltusotine may be alternatively characterized by an X-ray powder diffraction pattern having peaks 9.7, 10.8, 15.6, 17.3, 18.9, 19.6, 22.8 and 29.3 degrees 2-theta±0.2 degrees 2-theta.

In one embodiment of the present disclosure, crystalline Form PL14 of Paltusotine is isolated.

Crystalline Form PL14 of Paltusotine may be characterized by each of the above characteristics alone/or by all possible combinations, e.g., an XRPD pattern having peaks at 9.7, 10.8, 15.6, 18.9 and 29.3 degrees 2-theta±0.2 degrees 2-theta; an XRPD pattern as depicted in FIG. 16, and combinations thereof.

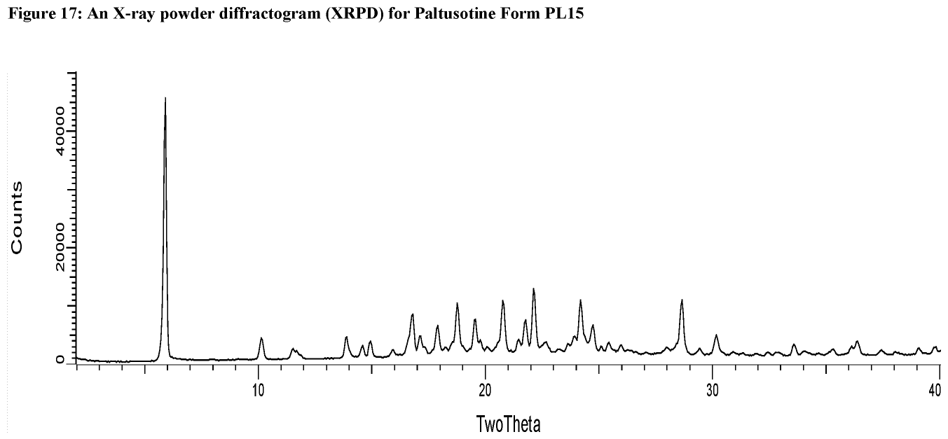

The present disclosure includes a crystalline polymorph of Paltusotine, designated PL15. The crystalline Form PL15 of Paltusotine may be characterized by data selected from one or more of the following: an X-ray powder diffraction pattern substantially as depicted in FIG. 17; an X-ray powder diffraction pattern having peaks at 6.0, 16.9, 18.9, 22.2 and 28.7 degrees 2-theta±0.2 degrees 2-theta; and combinations of these data.

Crystalline Form PL15 of Paltusotine may be further characterized by an X-ray powder diffraction pattern having peaks at 6.0, 16.9, 18.9, 22.2 and 28.7 degrees 2-theta±0.2 degrees 2-theta, and also having any one, two, three, or four additional peaks selected from 10.2, 14.0, 19.6 and 30.3 degrees 2-theta±0.2 degrees 2-theta.

Crystalline Form PL15 of Paltusotine may be alternatively characterized by an X-ray powder diffraction pattern having peaks 6.0, 10.2, 14.0, 16.9, 18.9, 19.6, 22.2, 28.7 and 30.3 degrees 2-theta±0.2 degrees 2-theta.

In one embodiment of the present disclosure, crystalline Form PL15 of Paltusotine is isolated.

Crystalline Form PL15 of Paltusotine may be characterized by each of the above characteristics alone/or by all possible combinations, e.g., an XRPD pattern having peaks at 6.0, 16.9, 18.9, 22.2 and 28.7 degrees 2-theta±0.2 degrees 2-theta; an XRPD pattern as depicted in FIG. 17, and combinations thereof.

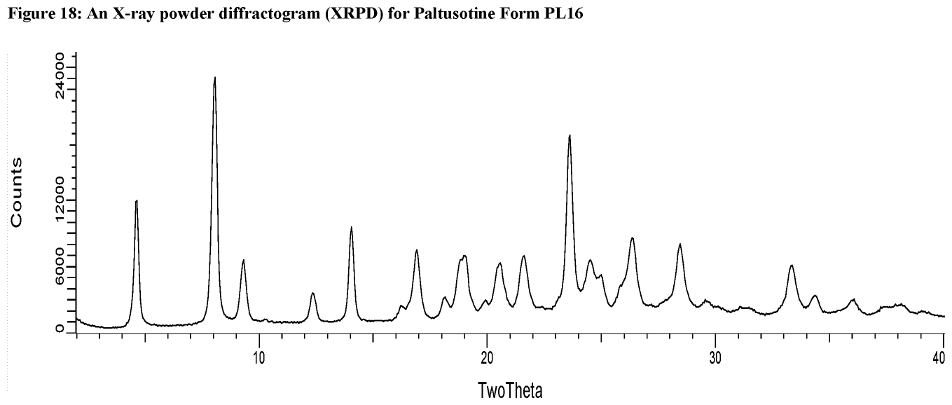

The present disclosure includes a crystalline polymorph of Paltusotine, designated PL16. The crystalline Form PL16 of Paltusotine may be characterized by data selected from one or more of the following: an X-ray powder diffraction pattern substantially as depicted in FIG. 18; an X-ray powder diffraction pattern having peaks at 4.7, 8.1, 14.1, 21.7 and 23.7 degrees 2-theta±0.2 degrees 2-theta; and combinations of these data.

Crystalline Form PL16 of Paltusotine may be further characterized by an X-ray powder diffraction pattern having peaks at 4.7, 8.1, 14.1, 21.7 and 23.7 degrees 2-theta±0.2 degrees 2-theta, and also having any one, two, three or four additional peaks selected from 9.4, 12.4, 20.6 and 28.5 degrees 2-theta±0.2 degrees 2-theta.

Crystalline Form PL16 of Paltusotine may be alternatively characterized by an X-ray powder diffraction pattern having peaks 4.7, 8.1, 9.4, 12.4, 14.1, 20.6, 21.7, 23.7 and 28.5 degrees 2-theta±0.2 degrees 2-theta.

In one embodiment of the present disclosure, crystalline Form PL16 of Paltusotine is isolated.

Crystalline Form PL16 of Paltusotine may be characterized by each of the above characteristics alone/or by all possible combinations, e.g., an XRPD pattern having peaks at 4.7, 8.1, 14.1, 21.7 and 23.7 degrees 2-theta±0.2 degrees 2-theta; an XRPD pattern as depicted in FIG. 18, and combinations thereof.

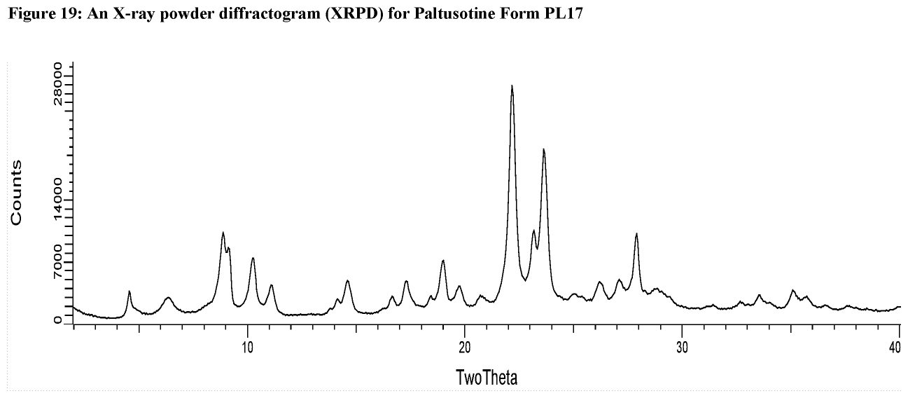

The present disclosure includes a crystalline polymorph of Paltusotine, designated PL17. The crystalline Form PL17 of Paltusotine may be characterized by data selected from one or more of the following: an X-ray powder diffraction pattern substantially as depicted in FIG. 19; an X-ray powder diffraction pattern having peaks at 10.3, 17.4, 19.1, 22.3 and 28.0 degrees 2-theta±0.2 degrees 2-theta; and combinations of these data.

Crystalline Form PL17 of Paltusotine may be further characterized by an X-ray powder diffraction pattern having peaks at 10.3, 17.4, 19.1, 22.3 and 28.0 degrees 2-theta±0.2 degrees 2-theta, and also having any one, two, three or four additional peaks selected from 9.0, 19.8, 23.7 and 26.3 degrees 2-theta±0.2 degrees 2-theta.

Crystalline Form PL17 of Paltusotine may be alternatively characterized by an X-ray powder diffraction pattern having peaks 9.0, 10.3, 17.4, 19.1, 19.8, 22.3, 23.7, 26.3 and 28.0 degrees 2-theta±0.2 degrees 2-theta.

In one embodiment of the present disclosure, crystalline Form PL17 of Paltusotine is isolated.

Crystalline Form PL17 of Paltusotine may be characterized by each of the above characteristics alone/or by all possible combinations, e.g., an XRPD pattern having peaks at 10.3, 17.4, 19.1, 22.3 and 28.0 degrees 2-theta±0.2 degrees 2-theta; an XRPD pattern as depicted in FIG. 19, and combinations thereof.

The crystalline polymorphs of any aspect or embodiment of the disclosure may be polymorphically pure and/or chemically pure as described herein.

The present disclosure also encompasses the use of crystalline polymorphs of Paltusotine and salts of Paltusotine, particularly Paltusotine monomesylate and Paltusotine hemimesylate of the present disclosure for the preparation of pharmaceutical compositions of crystalline polymorph Paltusotine and/or crystalline polymorphs thereof.

The present disclosure includes processes for preparing the above mentioned pharmaceutical compositions. The processes includes combining any one or a combination of the crystalline polymorphs of Paltusotine and salts of Paltusotine, particularly Paltusotine monomesylate and Paltusotine hemimesylate of the present disclosure with at least one pharmaceutically acceptable excipient.

Pharmaceutical combinations or formulations of the present disclosure contain any one or a combination of the solid state forms of Paltusotine and salts of Paltusotine, particularly Paltusotine monomesylate and Paltusotine hemimesylate of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Paltusotine and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Paltusotine can be administered. Paltusotine may be formulated for administration to a mammal, in embodiments to a human, by injection. Paltusotine can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Paltusotine and the pharmaceutical compositions and/or formulations of Paltusotine of the present disclosure can be used as medicaments, in embodiments in the treatment of acromegaly, malignant carcinoid syndrome and neuroendocrine tumours.

The present disclosure also provides methods of treating acromegaly, malignant carcinoid syndrome and neuroendocrine tumours by administering a therapeutically effective amount of any one or a combination of the crystalline polymorphs of Paltusotine of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

X-ray diffraction was performed on X-Ray powder diffractometer: Bruker D8 Advance; CuKα radiation (λ=1.5418 Å); Lynx eye detector; laboratory temperature 22-25° C.; PMMA specimen holder ring with silicon low background. Prior to analysis, the samples were gently ground by means of mortar and pestle in order to obtain a fine powder. The ground sample was adjusted into a cavity of the sample holder and the surface of the sample was smoothed by means of a cover glass.

### Measurement Parameters:

Scan range: 2-40 degrees 2-theta;
Scan mode: continuous;
Step size: 0.05 degrees;
Time per step: 0.5 s;
Sample spin: 30 rpm;
Sample holder: PMMA specimen holder ring with silicon low background.

All X-Ray Powder Diffraction peak values are calibrated with regard to standard silicon spiking in the sample.

Solid State ¹³C-NMR (¹³C CP/MAS NMR) Method

Solid-state NMR spectra were measured at 11.7 T using a Bruker Avance III HD 500 US/WB NMR spectrometer (Karlsruhe, Germany, 2013) with 3.2 mm probehead. The ¹³C CP/MAS NMR spectra employing cross-polarization were acquired using the standard pulse scheme at spinning frequency of 18 kHz and a room temperature (300 K). The recycle delay was 8 s and the cross-polarization contact time was 2 ms. The ¹³C scale was referenced to α-glycine (176.03 ppm for ¹³C). Frictional heating of the spinning samples was offset by active cooling, and the temperature calibration was performed with Pb(NO₃)₂. The NMR spectrometer was completely calibrated and all experimental parameters were carefully optimized prior the investigation. Magic angle was set using KBr during standard optimization procedure and homogeneity of magnetic field was optimized using adamantane sample (resulting line-width at half-height Δν1/2 was less than 3.5 Hz at 250 ms of acquisition time).

### EXAMPLES

### Preparation of Starting Materials

Paltusotine free base can be prepared according to methods provided in WO2019/143718; or alternatively according to Example 27 below. The Paltusotine free base prepared according to these methods may be used as starting materials in the examples below. Particularly, the Paltusotine free base may be crystalline Pattern A as described in WO2019/143718. Paltusotine dimesylate can be prepared according to methods known from the literature, for example WO2021/011641.

A pH 6.8 buffer may be prepared according any suitable method, particularly in accordance with the US Pharmacopoeia (USP 35-NF 30 Buffer solutions). A suitable buffer can be prepared using KH₂PO₄ solution and sodium hydroxide solution:

The following Solutions 1 and 2 were prepared:
Solution 1: 13.61 g of KH₂PO₄ in 500 mL
Solution 2: 1.61 g of NaOH in 200 mL

Then, 250 mL of Solution 1 and 114 mL of Solution 2 were mixed together were combined. Water was added to the combined solutions to make up to a total volume of 1000 ml. This solution can be used as the pH 6.8 buffer in the examples.

### Example 1: Preparation of Paltusotine Form PL1

Paltusotine Pattern A (0.03 g) was taken in a 2 mL vial and was suspended in 1 mL of Propionitrile at temperature of about 60° C. The suspension was stirred for 1 day and filtered under vacuum at temperature of about 25° C. Further the solid was dried under vacuum at temperature of about 60° C. for period of about 2 hours. The obtained solid was analyzed by XRPD. Crystalline Paltusotine Form PL1 was obtained. An XRPD pattern is shown in FIG. 1.

### Example 2: Preparation of Paltusotine Form PL1

Paltusotine Pattern A (0.03 g) was taken in a 2 mL vial and was suspended in 1 mL of Cyclohexane at temperature of about 60° C. The suspension was stirred for period of about 1 day and filtered under vacuum at temperature of about 25° C. Further the solid was dried under vacuum at temperature of about 60° C. for period of about 2 hours. The obtained solid was analyzed by XRPD. Crystalline Paltusotine Form PL1 was obtained.

### Example 3: Preparation of Paltusotine Form PL1

Paltusotine Pattern A (0.03 g) was taken in a 2 mL vial and was suspended in 1 mL of Cyclopentyl methyl ether at temperature of about 60° C. The suspension was stirred for period of about 1 day and filtered under vacuum at temperature of about 25° C. Further the solid was dried under vacuum at temperature of about 60° C. for period of about 2 hours. The obtained solid was analyzed by XRPD. Crystalline Paltusotine Form PL1 was obtained.

### Example 4: Preparation of Paltusotine Form PL1

Paltusotine Pattern A (0.05 g) was taken in a 2 mL vial and was suspended in 1 mL of Ethyl acetate at temperature of about 60° C. The suspension stirred for period of about 1 day and filtered under vacuum at temperature of about 25° C. Further the solid was dried under vacuum at temperature of about 60° C. for period of about 2 hours. The obtained solid was analyzed by XRPD. Crystalline Paltusotine Form PL1 was obtained.

### Example 5: Preparation of Paltusotine Form PL1

Paltusotine Pattern A (0.1 g) was suspended in 3 mL of IPAC at temperature of about 25° C. The suspension was stirred for period of about 48 hours and filtered under vacuum at temperature of about 25° C. The solid was dried under vacuum at temperature of about 60° C. for period of about 2 hours. The obtained solid was analyzed by XRPD. Crystalline Paltusotine Form PL1 was obtained.

### Example 6: Preparation of Paltusotine Form PL11

Paltusotine Form PL1 (0.05 g) was suspended in 2 mL of MTBE and stirred for period of about 48 hours at temperature of about 25° C. The obtained solid was filtered under vacuum at temperature of about 25° C. and dried under vacuum at temperature of about 60° C. for period of about 2 hours. The obtained solid was analyzed by XRPD. Crystalline Paltusotine Form PL11 was obtained. An XRPD pattern is shown in FIG. 2.

### Example 7: Preparation of Paltusotine Form PL11

Paltusotine Pattern A (0.1 g) was suspended in buffer solution* (pH=6.8; 1 mL) and stirred for period of about 48 hours at temperature of about 25° C. The obtained solid was filtered under vacuum at temperature of about 25° C. The wet solid was further suspended in 1 mL of water and stirred for period of about 24 hours. The solid was filtered under vacuum at temperature of about 25° C. and dried under vacuum at temperature of about 60° C. for period of about 2 hours. The obtained solid was analyzed by XRPD. Crystalline Paltusotine Form PL11 was obtained. *as per USP

### Example 8: Preparation of Paltusotine Form PL11

Paltusotine (0.03 g) was dissolved in 0.3 mL of Acetic acid at temperature of about 25° C. The clear solution was added to 2 mL of Ethyl acetate which was kept at temperature of about 25° C. and solution was maintained at temperature of about 25° C. with stirring for period of about 48 hours. The obtained solid was filtered under vacuum at temperature of about 25° C. The wet solid was further suspended in 1 mL of water and stirred for period of about 24 hours. The solid was filtered under vacuum at 25° C. and dried under vacuum at temperature of about 60° C. for period of about 2 hours. The obtained solid was analyzed by XRPD. Crystalline Paltusotine Form PL11 was obtained.

### Example 9: Preparation of Paltusotine Monomesylate Form PLM1

Paltusotine dimesylate (0.25 g) was dissolved in 1 mL of N-methyl Formamide and heated to temperature of about 70° C. for period of about 20 minutes. The clear solution was filtered with 0.45 micron filter. The hot solution was added to 3 ml of Acetonitrile anti-solvent which was kept at 25° C. Solid was obtained immediately and was stirred for period of about 15 minutes at temperature of about 25° C. The solid obtained was filtered and dried under vacuum at 25° C. for period of about 10 minutes, and further dried under vacuum tray drier at temperature of about 60° C. for period of about 1 hour. The obtained solid was analyzed by XRPD. Crystalline Paltusotine monomesylate Form PLM1 was obtained. An XRPD pattern is shown in FIG. 3.

### Example 10: Preparation of Paltusotine Monomesylate Form PLM1

Paltusotine dimesylate (0.6 g) was dissolved in 2 mL of N-methyl Formamide and heated to temperature of about 70° C. for period of about 20 minutes. The clear solution was filtered with 0.45 micron filter. 0.06 mL of the hot solution was added to 3 ml of 2-Butoxyethanol anti-solvent which was kept at 25° C. Solid was obtained immediately and was stirred for period of about 15 minutes at temperature of about 25° C. The solid obtained was filtered and dried under vacuum at 25° C. for period of about 10 minutes. The obtained solid was analyzed by XRPD. Crystalline Paltusotine monomesylate Form PLM1 was obtained.

### Example 11: Preparation of Paltusotine Hemimesylate Form PLM2

Paltusotine (0.5 g) (Form PL1) was taken in a 50 mL RB and was suspended in 10 mL of Acetone at temperature of about 50° C. for period of about 30 minutes. Then 100 uL Methane Sulfonic Acid (MSA) was added dropwise at temperature of about 50° C. The suspension was stirred for period of about 4 Hours and was filtered under vacuum at temperature of about 25° C. The obtained solid analyzed by XRPD. Crystalline Paltusotine hemimesylate Form PLM2 was obtained. An XRPD pattern is shown in FIG. 4.

### Example 12: Preparation of Paltusotine Form PL2

Paltusotine Pattern A (0.05 g) was taken in a 2 mL vial and was suspended in 1 mL of Acetonitrile at temperature of about 60° C. The suspension was stirred for period of about 1 day and filtered under vacuum at temperature of about 25° C. The obtained solid was analyzed by XRPD. Crystalline Paltusotine Form PL2 was obtained. An XRPD pattern is shown in FIG. 5.

### Example 13: Preparation of Paltusotine Form PL3

Paltusotine Pattern A (0.03 g) was taken in a 2 mL vial and was suspended in 1 mL of 1-butanol at temperature of about 60° C. The suspension was stirred for period of about 1 day and filtered under vacuum at temperature of about 25° C. Further the solid was dried under vacuum at temperature of about 60° C. for period of about 2 hours. The obtained solid was analyzed by XRPD. Crystalline Paltusotine Form PL3 was obtained. An XRPD pattern is shown in FIG. 6.

### Example 14: Preparation of Paltusotine Form PL4

Paltusotine Pattern A (0.03 g) was taken in a 2 mL vial and was suspended in 1 mL of Diethylketone at temperature of about 60° C. The suspension was stirred for period of about 1 day and filtered under vacuum at temperature of about 25° C. Further the solid was dried under vacuum at temperature of about 60° C. for period of about 2 hours. The obtained solid was analyzed by XRPD. Crystalline Paltusotine Form PL4 was obtained. An XRPD pattern is shown in FIG. 7.

### Example 15: Preparation of Paltusotine Form PL5

Paltusotine Pattern A (0.03 g) was taken in a 2 mL vial and was suspended in 1 mL of 1,2-Dimethoxyethane at temperature of about 60° C. The suspension stirred for period of about 1 day and filtered under vacuum at temperature of about 25° C. Further the solid was dried under vacuum at temperature of about 60° C. for period of about 2 hours. The obtained solid analyzed by XRPD. Crystalline Paltusotine Form PL5 was obtained. An XRPD pattern is shown in FIG. 8.

### Example 16: Preparation of Paltusotine Form PL6

Paltusotine Pattern A (0.05 g) was taken in a 2 mL vial and was suspended in 1 mL of Hexanol at temperature of about 60° C. The suspension stirred for period of about 1 day and filtered under vacuum at temperature of about 25° C. Further the solid was dried under vacuum at temperature of about 60° C. for period of about 2 hours. The obtained solid analyzed by XRPD. Crystalline Paltusotine Form PL6 was obtained. An XRPD pattern is shown in FIG. 9.

### Example 17: Preparation of Paltusotine Form PL7

Paltusotine Pattern A (0.05 g) was taken in a 2 mL vial and was suspended in 1 mL of Methyl Acetate at temperature of about 60° C. The suspension was stirred for period of about 1 day and filtered under vacuum at temperature of about 25° C. Further the solid was dried under vacuum at temperature of about 60° C. for period of about 2 hours. The obtained solid analyzed by XRPD. Crystalline Paltusotine Form PL7 was obtained. An XRPD pattern is shown in FIG. 10.

### Example 18: Preparation of Paltusotine Form PL8

Paltusotine Pattern A (0.03 g) was taken in a 2 mL vial and was suspended in 1 mL of tert-amyl alcohol at temperature of about 60° C. The suspension stirred for period of about 1 day and filtered under vacuum at temperature of about 25° C. Further the solid was dried under vacuum at temperature of about 60° C. for period of about 2 hours. The obtained solid analyzed by XRPD. Crystalline Paltusotine Form PL8 was obtained. An XRPD pattern is shown in FIG. 11.

### Example 19: Preparation of Paltusotine Form PL9

Paltusotine Pattern A (0.05 g) was taken in a 2 mL vial and was suspended in 1 mL of 1,4-Dioxane at temperature of about 60° C. The suspension stirred for period of about 1 day and filtered under vacuum at period of about 25° C. Further the solid was dried under vacuum at temperature of about 60° C. for 2 hours. The obtained solid analyzed by XRPD. Crystalline Paltusotine Form PL9 was obtained. An XRPD pattern is shown in FIG. 12.

### Example 20: Preparation of Paltusotine Form PL10

Paltusotine Pattern A (0.03 g) was taken in a 2 mL vial and was suspended in 1 mL of Isobutanol at temperature of about 60° C. The suspension stirred for period of about 1 day and filtered under vacuum at temperature of about 25° C. Further the solid was dried under vacuum at temperature of about 60° C. for period of about 2 hours. The obtained solid analyzed by XRPD. Crystalline Paltusotine Form PL10 was obtained. An XRPD pattern is shown in FIG. 13.

### Example 21: Preparation of Paltusotine Form PL12

Paltusotine free base (0.02 g) was dissolved in 0.75 mL of DMSO at temperature of about 60° C. The clear solution was added to 2 mL of MTBE which was kept at temperature of about 25° C. and solution was maintained at temperature of about 25° C. with stirring for period of about 24 hours. Solid obtained was filtered and dried suction at temperature of about 25° C. for period of about 15 minutes. The obtained solid was analyzed by XRPD. Crystalline Paltusotine Form PL12 was obtained. An XRPD pattern is shown in FIG. 14.

### Example 22: Preparation of Paltusotine Form PL13

Paltusotine free base (0.02 g) was dissolved in 1.5 mL of Acetone with 4% of DMSO at temperature of about 60° C. The clear solution was added to 3 mL of Isopropyl acetate which was kept at temperature of about 25° C. and solution was maintained at temperature of about 25° C. with stirring for period of about 48 hours. Solid obtained was filtered and dried suction at temperature of about 25° C. for period of about 15 minutes. The obtained solid was analyzed by XRPD. Crystalline Paltusotine Form PL13 was obtained. An XRPD pattern is shown in FIG. 15.

### Example 23: Preparation of Paltusotine Form PL14

Paltusotine free base (0.02 g) was dissolved in 0.75 mL of DMSO at temperature of about 60° C. The clear solution was added to 2 mL of Acetonitrile which was kept at temperature of about 25° C. and solution was maintained at temperature of about 25° C. with stirring for period of about 48 hours. Solid obtained was filtered and dried suction at temperature of about 25° C. for period of about 15 minutes. The obtained solid was analyzed by XRPD. Crystalline Paltusotine Form PL14 was obtained. An XRPD pattern is shown in FIG. 16.

### Example 24: Preparation of Paltusotine Form PL15

Paltusotine free base (0.03 g) was dissolved in 0.3 mL of Benzyl alcohol at temperature of about 25° C. The clear solution was added to 2 mL of Cyclohexane which was kept at temperature of about 25° C. and solution was maintained at temperature of about 25° C. with stirring for period of about 24 hours. Solid obtained was filtered and dried suction at temperature of about 25° C. for period of about 15 minutes. The obtained solid was analyzed by XRPD. Crystalline Paltusotine Form PL15 was obtained. An XRPD pattern is shown in FIG. 17.

### Example 25: Preparation of Paltusotine Form PL16

Paltusotine free base (0.03 g) was dissolved in 0.3 mL of Acetic acid at temperature of about 25° C. The clear solution was added to 2 mL of Ethyl acetate which was kept at temperature of about 25° C. and solution was maintained at temperature of about 25° C. with stirring for period of about 48 hours. Solid obtained was filtered and dried suction at temperature of about 25° C. for period of about 15 minutes. The obtained solid was analyzed by XRPD. Crystalline Paltusotine Form PL16 was obtained. An XRPD pattern is shown in FIG. 18.

### Example 26: Preparation of Paltusotine Form PL17

Paltusotine free base (0.03 g) was dissolved in 0.25 mL of Acetone and Formic acid mixture (1:1) at temperature of about 25° C. The clear solution was added to 2 mL of Ethyl acetate which was kept at temperature of about 25° C. and solution was maintained at temperature of about 25° C. with stirring for period of about 48 hours. Solid obtained was filtered and dried suction at temperature of about 25° C. for period of about 15 minutes. The obtained solid was analyzed by XRPD. Crystalline Paltusotine Form PL17 was obtained. An XRPD pattern is shown in FIG. 19.

### Example 27: Preparation of Paltusotine Free Base (Pattern A)

Paltusotine free base (0.02 g) was dissolved in 1.5 mL of 4% DMSO in acetone at 60° C. The clear solution added into isopropyl acetate (3 mL) at 25° C. and maintained under stirring at 25° C. for 48 hours. Solid obtained was filtered and wet solid was further suspended in 1 mL of water at 60° C. for 12 hours. Filtered and dried under vacuum at 25° C. for about 15 minutes and then dried under vacuum at 60° C. for 2 hours.

### Example 28: Stability Experiments

### Storage Stability at Different Relative Humidities

Samples of Paltusotine forms PL1 and PL11 were subjected to conditions of different relative humidities at ambient temperature. XRPD analysis was performed on the samples after 7 days. The results are shown in Table 1:

TABLE 1

| XRPD | | | | | |
|---|---|---|---|---|---|
| analysis | Relative humidity | | | | |
| results | 20% | 40% | 60% | 80% | 100% |
| Form PL1 | No | No | No | No | No |
| | change | change | change | change | change |
| Form PL11 | No | No | No | No | No |
| | change | change | change | change | change |

These results demonstrate that Paltusotine forms PL1 and PL11 are stable after exposure to high and low relative humidity for at least 7 days.

Samples of Paltusotine form PL1 was subjected to conditions of different relative humidities at different temperatures. XRPD analysis was performed on the samples after 6 months. The results are shown in Table 2:

TABLE 2

| | |
|---|---|
| XRPD | Conditions (6 months) |

| analysis | 25° C., | 40° C., |
|---|---|---|
| results | 60% RH | 75% RH |
| Form PL1 | No change | No change |

The results demonstrate that Paltusotine forms PL1 is stable after exposure to high and low relative humidity at different temperatures for at least 6 months, indicating that this form has excellent storage stability.

### Grinding Experiments

Samples of Paltusotine forms PL1 and PL11 were subjected to strong grinding, and to solvent drop grinding in water, ethanol and isopropanol. Grinding was carried out on the samples alone, or in the presence of ethanol or water. In these experiments, about 20 mg of the sample is placed in a mortar and ground with a pestle for 2 minutes. The solvent, when used, as added to the crystalline material before grinding, in a volume of 10 microlitres. XRPD analysis performed on each of the samples after the grinding experiment, confirmed no change in the starting material (Table 3):

TABLE 3

| | Form PL1 | Form PL11 |
|---|---|---|
| | XRPD analysis | XRPD analysis |
| Condition | results | results |
| Strong grinding | No change | No change |
| Solvent-drop grinding | No change | No change |
| (ethanol) | | |
| Solvent-drop grinding (water) | No change | No change |
| Solvent-drop grinding | No change | No change |
| (isopropanol) | | |

The results demonstrate that Paltusotine forms PL1 and PL11 is resistant to polymorphic changes and is highly suitable for preparing pharmaceutical formulations.

### Thermal Stability

Samples of Paltusotine forms PL1 and PL11 were subjected to heating up to 100° C. for 30 minutes. XRPD analysis of the sample confirmed no change in the starting material (Table 4):

TABLE 4

| | Form PL1 | Form PL11 |
|---|---|---|
| Condition | Result | Result |
| Heating 100° C., 30 minutes | Stable | Stable |

### Stability to Compression

Samples of Paltusotine forms PL1 and PL11 were subjected to a pressure of 2 tons in a hydraulic press (Atlas® Autopress hydraulic press, set to 2 tons). XRPD analysis was performed on the samples after 1 minute. The results are shown in Table 5:

TABLE 5

| | Form PL1 | Form PL11 |
|---|---|---|
| Condition | Result | Result |
| Pressure, 2 tons/1 | Stable | Stable |
| minute | | |

These results further confirm the stability of Paltusotine forms PL1 and PL11, and demonstrate their suitability for pharmaceutical processing.

1. A crystalline Form PL1 of Paltusotine characterized by data including at least one of the following:
a. an XRPD pattern having peaks at 6.3, 9.5, 14.8, 22.9 and 27.6 degrees 2-theta±0.2 degrees 2-theta;
b. an XRPD pattern as depicted in FIG. 1;
c. a solid state ¹³C NMR spectrum having peaks at 33.2, 116.7, 129.4, 142.1, 148.7 and 167.7 ppm±0.2 ppm;
d. a solid state ¹³C NMR spectrum having the following chemical shift absolute differences from a reference peak at 102.6 ppm±2 ppm of 69.4, 14.1, 26.8, 39.5, 46.1 and 65.1 ppm±0.1 ppm; or
e. a solid-state ¹³C NMR spectrum substantially as depicted in FIG. 20a, 20b or 20c.

2. The crystalline Form PL1 of Paltusotine according to claim 1, which is characterized by an XRPD pattern having peaks at 6.3, 9.5, 14.8, 22.9 and 27.6 degrees 2-theta±0.2 degrees 2-theta, and also having one, two, three, or four additional peaks selected from 11.5, 13.3, 19.9 and 22.1 degrees two theta±0.2 degrees two theta.

3. The crystalline Form PL1 of Paltusotine according to claim 1, which is characterized by an XRPD pattern having peaks at: 6.3, 9.5, 11.5, 13.3, 14.8, 19.9, 22.1, 22.9 and 27.6 degrees 2-theta±0.2 degrees 2-theta.

4. The crystalline Form PL1 of Paltusotine according to claim 1, wherein said crystalline form is a hydrate form.

5. A crystalline Form PL11 of Paltusotine characterized by data including at least one of the following:
a. an XRPD pattern having peaks at 4.5, 7.1, 15.2, 18.6 and 21.7 degrees 2-theta±0.2 degrees 2-theta;
b. an XRPD pattern as depicted in FIG. 2;
c. a solid state ¹³C NMR spectrum having peaks at 44.8, 51.7, 135.4, 147.5, 150.2 and 154.4 ppm±0.2 ppm;
d. a solid state ¹³C NMR spectrum having the following chemical shift absolute differences from a reference peak at 102.6 ppm±2 ppm of 57.8, 50.9, 32.8, 44.9, 47.6 and 51.8 ppm±0.1 ppm; or
e. a solid-state ¹³C NMR spectrum substantially as depicted in FIG. 21a, 21b or 21c.

6. The crystalline Form PL11 of Paltusotine according to claim 5, which is characterized by an XRPD pattern having peaks at 4.5, 7.1, 15.2, 18.6 and 21.7 degrees 2-theta±0.2 degrees 2-theta, and also having one, two, three, or four additional peaks selected from 11.4, 18.2, 28.1 and 30.7 degrees two theta±0.2 degrees two theta.

7. The crystalline Form PL11 of Paltusotine according to claim 5, which is characterized by an XRPD pattern having peaks at: 4.5, 7.1, 11.4, 15.2, 18.2, 18.6, 21.7, 28.1 and 30.7 degrees 2-theta±0.2 degrees 2-theta.

8. The crystalline Form PL11 of Paltusotine according to claim 5, wherein said crystalline form is a hydrate form.

9. The crystalline Form PL1 of Crystalline Paltusotine according to claim 5, which contains: no more than about 20%, no more than about 10%, no more than about 5%, no more than about 2%, no more than about 1% or about 0% of any other crystalline forms of Paltusotine.

10. The crystalline Form PL1 of Paltusotine according to claim 1, which contains: no more than about 20%, no more than about 10%, no more than about 5%, no more than about 2%, no more than about 1% or about 0% of amorphous Paltusotine.

11. A pharmaceutical composition comprising the crystalline Form PL1 of Paltusotine according to claim 1.

12. (canceled)

13. The pharmaceutical composition according to claim 11, with at least one pharmaceutically acceptable excipient.

14. A process for preparing a pharmaceutical formulation, the process comprising: combining the crystalline Form PL1 of Paltusotine according to claim 1, with at least one pharmaceutically acceptable excipient.

15. (canceled)

16. A method of treating a condition, comprising administering a therapeutically effective amount of the crystalline Form PL1 of Paltusotine according to claim 1, in the treatment of at least one of acromegaly, malignant carcinoid syndrome, or neuroendocrine tumours.

17. (canceled)

18. (canceled)

19. A process comprising:
preparing another solid state form of Paltusotine, or another Paltusotine salt or solid state form thereof from the crystalline Form PL1 of Paltusotine, according to claim 1.

20. The crystalline Form PL11 of Paltusotine according to claim 5, which contains: no more than about 20%, no more than about 10%, no more than about 5%, no more than about 2%, no more than about 1% or about 0% of amorphous Paltusotine.

21. The crystalline Form PL11 of Paltusotine according to claim 5, which contains: no more than about 20%, no more than about 10%, no more than about 5%, no more than about 2%, no more than about 1% or about 0% of any other crystalline forms of Paltusotine.

22. A pharmaceutical composition comprising the crystalline Form PL11 of Paltusotine according to claim 5.

23. The pharmaceutical composition according to claim 22, with at least one pharmaceutically acceptable excipient.

24. A process for preparing a pharmaceutical formulation, the process comprising: combining the crystalline Form PL11 of Paltusotine according to claim 5 with at least one pharmaceutically acceptable excipient.

25. A method of treating a condition, comprising administering a therapeutically effective amount of the crystalline Form PL11 of Paltusotine according to claim 5, in the treatment of at least one of acromegaly, malignant carcinoid syndrome, or neuroendocrine tumours.

26. A process comprising:
preparing another solid state form of Paltusotine, or another Paltusotine salt or solid state form thereof from the crystalline Form PL11 of Paltusotine, according to claim 5.
